(19) United States
(12) Patent Application Publication
(10) Pub. No.: US 2015/0030684 A1
(43) Pub. Date: Jan. 29, 2015

(54) IRRIGATION RESISTANT COMPOSITIONS FOR REGENERATION OF HARD TISSUES AND METHODS AND KITS OF USING THE SAME

(71) Applicants: Gregory J. Pomrink, Newberry, FL (US); Cecilia A. Cao, Gainesville, FL (US); Zehra Tosun, Gainesville, FL (US); David M. Gaisser, Gainesville, FL (US)

(72) Inventors: Gregory J. Pomrink, Newberry, FL (US); Cecilia A. Cao, Gainesville, FL (US); Zehra Tosun, Gainesville, FL (US); David M. Gaisser, Gainesville, FL (US)

(21) Appl. No.: 14/512,976

(22) Filed: Oct. 13, 2014

Related U.S. Application Data
(63) Continuation-in-part of application No. 14/369,119.
(60) Provisional application No. 61/738,585, filed on Dec. 18, 2012.
(60) Provisional application No. 61/787,827, filed on Mar. 15, 2013.

(51) Int. Cl. A61L 27/50; A61L 27/18; A61L 27/10; A61M 1/00; A61B 17/88; A61B 19/02; A61M 39/10; A61L 27/54; A61L 27/20

(52) U.S. Cl. CPC A61L 27/505; A61L 27/54; A61L 27/18; A61L 27/10; A61L 27/50; A61L 27/20; A61B 17/8822; A61B 19/026; A61M 39/10; A61M 1/008; A61L 2400/06; A61L 2300/60; A61L 2300/622; A61L 2300/412; A61L 2300/112; A61M 2210/02
USPC 424/493; 514/772; 424/602; 424/489; 514/788; 514/785; 514/784; 606/93; 606/94

(57) ABSTRACT

An irrigation resistant bone repair composition including a biocompatible or bioactive bone repair material and a mixture of non-random poly(oxyalkylene) block copolymers is described. Also, methods for treating a bone having a bone gap or a bone defect with the composition including a biocompatible or bioactive bone repair material and a mixture of non-random poly(oxyalkylene) block copolymers are also provided. Also, kits including the irrigation resistant bone repair composition including a biocompatible or bioactive bone repair material and a mixture of non-random poly(oxyalkylene) block copolymers are described.

### RELATED APPLICATIONS

The present patent document is a continuation-in-part application of U.S. patent application Ser. No. 14/369,119, filed Jun. 26, 2014, which is §371 nationalization of International Application No. PCT/US2013/075741, filed Dec. 17, 2013, which claims the benefit of the filing date under 35 U.S.C. §119(e) of Provisional U.S. Patent Application Ser. Nos. 61/738,585, filed Dec. 18, 2012 and 61/787,827, filed Mar. 15, 2013, which are incorporated herein by reference in their entirety.

### BACKGROUND

Bone is a composite of collagen, cells, calcium hydroxyapatite crystals, and small quantities of other proteins of organic molecules that has unique properties of high strength, rigidity, and ability to adapt to varying loads. When bone injuries occur, it is necessary to fill voids or gaps in the bone as well as to encourage the repair and regeneration of bone tissue. There are many materials used today for the repair and regeneration of bone defects. For example, one material useful to encourage such repair and regeneration is bioactive glass.

Bioactive glass was originally developed in 1969 by L. Hench. Additionally, bioactive glasses were developed as bone replacement materials, with studies showing that bioactive glass can induce or aid in osteogenesis (Hench et al., J. Biomed. Mater. Res. 5:117-141 (1971)). Bioactive glass can form strong and stable bonds with bone (Piotrowski et al., J. Biomed. Mater. Res. 9:47-61 (1975)). Further, bioactive glass is not considered toxic to bone or soft tissue from studies of in vitro and in vivo models (Wilson et al., J. Biomed. Mater. Res. 805-817 (1981)). Exemplary bioactive glasses include 45S5, 45S5B1, 58S, and S70C30. The original bioactive glass, 45S5, is melt-derived. Sol-gel derived glasses can also be produced and include nanopores that allow for increased surface area and bioactivity.

There are drawbacks to the use of bioactive glass or other materials in the form of liquids, pastes, and solids to fill voids or gaps in the bone. A liquid or a paste may not remain at the site of the void or gap in the bone. A solid may be difficult to apply and may not conform well to the void or gap in the bone.

These drawbacks may be reduced and/or eliminated by adding materials to a bone repair composition, such that the composition is rendered irrigation resistant.

### BRIEF DESCRIPTION OF THE DRAWINGS

The patent or application file contains at least one drawing (color photographs) executed in color. Copies of this patent or patent application publication with color drawing(s) will be provided by the Office upon request and payment of the necessary fee.

FIG. 1 depicts an exemplary delivery system kit for delivering an irrigation resistant bone repair composition.

FIG. 2A-B depicts schematic drawings of an adapter (2A) and a delivery gun (2B) for the irrigation resistant bone repair composition.

FIG. 3 depicts a schematic drawing of a plunger of the delivery system.

FIG. 4A depicts exemplary tips for a delivery system.

FIG. 4B depicts exemplary tips for a delivery system.

FIG. 5A is a photograph of the tubes filled with an irrigation resistant bone repair composition for use with a delivery system.

FIG. 5B depicts a schematic drawing of a tube for use with a delivery system.

FIG. 6A is a photograph of an exemplary delivery system for an irrigation resistant bone repair composition.

FIG. 6B is a photograph of an exemplary delivery system for an irrigation resistant bone repair composition.

FIG. 7 is a photograph of an exemplary delivery system for an irrigation resistant bone repair composition.

FIG. 8 is a photograph of an exemplary delivery system for an irrigation resistant bone repair composition.

FIG. 9 is a photograph of an exemplary delivery system for an irrigation resistant bone repair composition.

FIG. 10 depicts IRM images of the histological stains.

FIG. 11 depicts a graph of compression and sustainability Results for the tested samples.

FIG. 12 depicts pictures from the sustainability testing of samples.

### SUMMARY

Certain embodiments relate to an irrigation resistant bone repair composition comprising a biocompatible bone repair material and a mixture of at least one non-random poly(oxyalkylene) block copolymer and at least one non-ionic surfactant other than the non-random poly(oxyalkylene) block copolymer. The non-ionic surfactant or similar material other than the non-random poly(oxyalkylene) block copolymer is selected from the group consisting of fatty alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 Monostearate), alkoxylated fatty ethers (e.g., polyethylene glycol lauryl ether (Brij L23), alkoxylated sorbitan esters (e.g., Span 85 (sorbitan trioleate)), alkoxylated sorbitan esters (e.g., Polysorbate 20 and PolySorbate 80 also referred to as Tween 20 and Tween 80), fatty acid esters or polyol esters (e.g., glycerol monostearate, PEG coconut triglycerides), polyalkylene glycols (e.g., PEG 400 and PEG 600). At least one of the surfactants in the composition has a melting point above room temperature, and more preferably above body temperature. The bone repair material can be any number of materials that assist in bone repair and production. Such materials include at least bioactive glass, spherical bioactive glass in a bimodal size distribution, and tricalcium phosphate, i.e., silicated tricalcium phosphate.

Further embodiments relate to bioactive glass particles including a coating comprising at least one poloxamer and at least one other surfactant, as well as a putty or paste including such poloxamer and other surfactant coated particles of bioactive glass.

Yet further embodiments relate to methods for treating a bone having a bone gap and/or a bone defect with the composition comprising a biocompatible bone repair material and a mixture of at least one poloxamer and at least one surfactant other than the non-random poly(oxyalkylene) block copolymer. The non-ionic surfactant or similar material other than the non-random poly(oxyalkylene) block copolymer is selected from the group consisting of fatty alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 Monostearate), alkoxylated fatty ethers (e.g., polyethylene glycol lauryl ether (Brij L23), alkoxylated sorbitan esters (e.g., Span 85 (sorbitan trioleate)), alkoxylated sorbitan esters (e.g., Polysorbate 20 and PolySorbate 80 also referred to as Tween 20 and Tween 80), fatty alcohols, fatty acids, fatty acid esters or polyol esters (e.g., glycerol monostearate, PEG coconut triglycerides), polyalkylene glycols (e.g., PEG 400 and PEG 600). At least one of the surfactants in the composition has a melting point above room temperature, and more preferably above body temperature.

Other embodiments relate to an irrigation resistant bone repair composition comprising a biocompatible bone repair material and a mixture of at least two nonrandom poly(oxyalkylene) block copolymers. The bone repair material can be any number of materials that assist in bone repair and production. Such materials include at least bioactive glass, spherical bioactive glass in a bimodal size distribution, and tricalcium phosphate, i.e., silicated tricalcium phosphate.

Further embodiments relate to bioactive glass particles including a coating comprising poloxamers, as well as a putty or paste including such poloxamer coated particles of bioactive glass.

Yet further embodiments relate to methods for treating a bone having a bone gap and/or a bone defect with the composition comprising a biocompatible bone repair material and a mixture of at least two nonrandom poly(oxyalkylene) block copolymers.

### DETAILED DESCRIPTION

An irrigation resistant bone repair composition comprising a biocompatible or bioactive bone repair material and a mixture of either at least one non-random poly(oxyalkylene) block copolymer and at least one non-ionic surfactant other than the non-random poly(oxyalkylene) block copolymer, or of two non-random poly(oxyalkylene) block copolymers is provided.

Specifically, certain embodiments relate to a synthetic bone grafting composition, such as a putty for bone repair that incorporates non-random ethylene oxide-propylene oxide block copolymers (in a class of compounds called poloxamers), having an osteoconductive, osteostimulative and irrigation resistant properties; i.e., the composition can be heavily irrigated in a surgical site without being washed away or displaced from the surgical site. The composition includes slow dissolving non-random block copolymers, which are mixed with a biocompatible or bioactive bone repair material, such as bioactive glasses or other osteoconductive salts, glasses or ceramics for use in methods for treating a bone having a bone gap and/or a bone defect.

The composition promotes osseointegration when introduced into a bone gap and/or a bone defect. The irrigation resistant characteristics provide a material, which maintains position in the surgical site despite the amount of blood, body fluid or saline to which it is exposed. Irrigation resistance is beneficial to simplify the application of the bone graft at the site of defect while preventing migration of the graft material during irrigation and after closure of the surgical site.

The bone repair composition has a unique physical property of being irrigation resistant. The irrigation resistance of the bone repair composition is especially beneficial for its intended use in orthopedic and spine processes, as the material will stabilize and maintain placement and structure within the body during placement, irrigation and after closure. Specifically, in certain embodiments where a non-setting putty material is used, the bone repair composition will not be displaced easily during irrigation and closure of the surgical site.

Furthermore, the bone repair composition is biocompatible and or bioactive and comprised of entirely synthetic materials, which fully eliminates any risk of disease transmission that may occur with other products containing animal or human derived materials or components to achieve this property.

As irrigation resistant, fully synthetic and bioactive putty, when implanted into the body, will maintain position or placement rather than melt to a liquid disintegrate during irrigation or displace upon closure of the surgical site. This feature permits the implant to hold in place more easily, and create beneficial handling properties. The ability to resist displacement allows more of the bioactive agent to remain at the site of implantation to stimulate bone growth for an extended period of time. The bioactive glass, as the preferred bioactive agent, stimulates the genes necessary to differentiate precursor cells into osteoblasts and the subsequent proliferation of these cells within the surgical site while undergoing an ionic exchange with the surrounding body fluid to form microcrystalline hydroxyapatite analogous to natural bone mineral. The combination of these properties in one composition is essential for bone regeneration and hard tissue repair.

In some embodiments, the composition is substantially a liquid at 5° C. and substantially a solid at 37° C. This effect can arise from the relative amount of poly(oxyalkylene) block copolymers in the composition, which in turn determines the viscosity of the composition at room temperature and at body temperature. For example, as the temperature rises, the composition becomes substantially more viscous to allow the bone repair material, for example bioactive glass, to more readily remain at the defect site.

The bone repair composition provides for acceleration in the rate and an enhancement in the quality of newly-formed bone. Improved bone healing may occur in those who may be compromised, such as diabetics, smokers, the obese, the elderly, those who have osteoporosis, those who use steroids, and those who have infections or other diseases that reduce the rate of healing. The rapid hardening of the bone repair composition at the site of the bone defect can serve to localize the bone repair material, such as bioactive glass, at the site.

The bone repair composition may be provided to a site of a bone defect by means of a syringe or other injection device. In certain embodiments, the bone repair composition may be sufficiently liquid so as to be injectable, yet can harden suitably at the bone defect site at body temperature. For instance, if the bone repair composition is a liquid at room temperature, it may become a thick gel at body temperature. Alternatively, it may be described that the bone repair composition cures upon application to a bone defect at body temperature.

In certain embodiments, the bone repair composition has the advantages of low viscosity, runny liquid composition with regard to the ease of application to a bone defect site. Further advantages of the composition include more solid paste-like composition characteristics and that it remains positioned at the defect after being applied. The solidification of the composition at body temperature overcomes the disadvantageous property of other liquid compositions that do not exhibit irrigation resistant behavior. At the same time, because the composition is not a solid at room temperature, there is greater ease of applying the composition, such as by means of a syringe. The composition need not be laboriously painted onto a bone defect or applied onto a bone defect by means of pressure.

Other delivery modes can be used for more viscous bone repair compositions. These modes include painting the gel or paste directly onto a bone defect or extruding the gel or paste as a bead.

In certain embodiments, if the bone repair composition is a gel at room temperature, it may become a paste at body temperature.

In certain other embodiments, if the bone repair composition is a thick gel or paste at room temperature, it may become putty or a solid at body temperature.

As noted above, the relative amount of poly(oxyalkylene) block copolymers in the composition will determine the viscosity at room temperature and at body temperature.

In certain embodiments, the irrigation resistant composition includes a biocompatible or bioactive bone repair material, and a mixture of at least one non-random poly(oxyalkylene) block copolymer and at least one non-ionic surfactant other than the non-random poly(oxyalkylene) block copolymer. The non-ionic surfactant or similar material other than the non-random poly(oxyalkylene) block copolymer is selected from the group consisting of fatty acids (e.g. stearic acid), fatty alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 Monostearate), alkoxylated fatty ethers (e.g., polyethylene glycol lauryl ether (Brij L23), alkoxylated sorbitan esters (e.g., Span 85 (sorbitan trioleate)), alkoxylated sorbitan esters (e.g., Polysorbate 20 and PolySorbate 80 also referred to as Tween 20 and Tween 80), fatty acid esters or polyol esters (e.g., glycerol monostearate, PEG coconut triglycerides), polyalkylene glycols (e.g., PEG 400 and PEG 600). Specific examples of surfactants other than the non-random poly(oxyalkylene) block copolymer include sorbitan tristearate, polysorbate 20, polysorbate 80, Polyoxyethylene 7 Coconut, Glycerides, PEG 400 Monostearate, PEG 2000 Monomethylether, and PEG 400 Distearate. At least one of the surfactants in the composition has a melting point above room temperature, and more preferably above body temperature.

In certain other embodiments, at least two poly(oxyalkylene) block copolymers may be included; alternatively, at least three or more poly(oxyalkylene) block copolymers may be included. In certain other embodiments, the irrigation resistant bone repair composition also includes at least two other surfactants; alternatively, at least three or more other surfactants are included.

In certain preferred embodiments, the irrigation resistant bone repair composition includes a mixture of at least two poly(oxyalkylene) block copolymers. In certain other embodiments, the irrigation resistant bone repair composition includes a mixture of 3, 4, 5 or more poly(oxyalkylene) block copolymers.

In various embodiments, the poly(oxyalkylene) block copolymers may be poloxamers. The poloxamer may be Poloxamer 407, Poloxamer 124, Poloxamer 188, Poloxamer 237, Poloxamer 338, and Poloxamer 407. The poly(oxyalkylene) block copolymer also is biocompatible, non-rigid, amorphous, and has no defined surfaces or three-dimensional structural features.

Poloxamers are non-random triblock copolymers composed of PEO and PPO units in the following structure: PEO-PPO-PEO. A particularly useful poloxamer in the context of the invention is Poloxamer 407 (Pluronic® F127). Poloxamer 407 has a high ratio of PEO to PPO and a high molar mass as compared to other poloxamers. The viscosity increases considerably as the temperature increases from 5° C. to 37° C. At a temperature below 25° C., a 20 wt % Poloxamer 407 solution behaves similarly to a viscous liquid while at body temperature (37° C.), the same solution behaves like a semisolid gel. Poloxamer 407 includes discrete blocks of both hydrophilic (oxyethylene) and hydrophobic (oxypropylene) subunits.

Non-random alkylene oxide copolymers, such as Poloxamer 407, have further advantages when used with bioactive glass over random copolymers. For example, non-random copolymers may be readily mixed in water to yield a thermoreversible composite whereas random copolymers alone cannot readily be formulated with water to yield a thermoreversible composite. The non-random poloxamers described herein may be formulated with bioactive glass and blood.

Poloxamer 407 is regarded as non-toxic. The biodegradability can be improved by using forms of Poloxamer 407 in which there are carbonate linkages incorporated into the structure.

The physical properties of Poloxamer 407 were extensively described in Li et al. (Li et al., “Thermoreversible micellization and gelation of a blend of Pluronic® polymers,” Polymer 49:1952-1960 (2008)), which is incorporated herein by reference in its entirety. The properties of Poloxamer 407 were also described in Lenaerts et al. (Lenaerts et al., “Temperature-dependent rheological behavior of Pluronic® F127 aqueous solutions,” International Journal of Pharmaceutics, 39: 121-127 (1987)), which is incorporated herein by reference in its entirety, and in Ivanova et al. (Ivanova et al., “Effect of Pharmaceutically Acceptable Glycols on the Stability of Liquid Crystalline Gels Formed by Poloxamer 407 in Water,” Journal of Colloid and Interface Science, 252: 226-235 (2002)), which is incorporated herein by reference in its entirety.

Another particularly useful poloxamer in the context of the invention is Poloxamer 124. Poloxamer 124 is also non-toxic and has been extensively studied (“Safety Assessment of Poloxamers 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, and 407, Poloxamer 105 Benzoate, and Poloxamer 182 Dibenzoate and Uses in Cosmetics,” International Journal of Toxicology, 27 (Suppl. 2):93-128, 2008; and Patel et al., “Poloxamers: A pharmaceutical excipients with therapeutic behaviors,” International Journal of PharmTech Research, 1(2):299-303, 2009).

The mixture of poloxamers with a bone growth factor material (i.e. BMP-2) was previously described by Rey-Rico et al. (Rey-Rico et al., “Osteogenic efficiency of in situ gelling poloxamine systems with and without bone morphogenetic protein-2,” European Cells and Materials, 21:317-340 (2011)), which is incorporated herein by reference in its entirety.

In certain embodiments, a mixture of at least two poly(oxyalkylene) block copolymers, such as a mixture of Poloxamer 407 and Poloxamer 124 may be formulated with a biocompatible or bioactive bone repair material.

Other poloxamers may also be used, provided that the poloxamers are substantially liquid at room temperature and have a higher viscosity at body temperature. Generally, such poloxamers have a high PEO content.

Specific examples of poloxamers that may be used in the irrigation resistant bone repair composition include Poloxamer P105, Poloxamer 124, Poloxamer 188, Poloxamer 237, and Poloxamer 338.

In certain embodiments, Poloxamer 407 may be combined with Poloxamer 124 and with a biocompatible or bioactive bone repair material.

In certain other embodiments, Poloxamer 105 or any other poloxamer may be combined with Poloxamer 407 or with any other poloxamer to obtain an optimal viscosity at both room temperature and body temperature.

Further, Poloxamer 407 or any other poloxamer used may be modified with means of adding functional groups. The functional groups may be, for example, hydroxyl end groups. Also, functional groups may have a positive charge such that the modified poloxamer is cationic.

In some embodiments, the weight ratio of the mixture of at least one poly(oxyalkylene) block copolymer and at least one non-ionic surfactant other than poly(oxyalkylene) block copolymer is 1%-99% relative to the weight of the bone repair composition. This weight ratio may be from 1-10%, 10-20%, 20-30%, 30%-40%, 40%-50%, 50%-60%, 60%-70%, 70%-80%, 80%-90%, or 90-99%. Alternatively, this weight ratio may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. The material may have the consistency of a solid, gel, putty, or any other non-liquid substance at room temperature.

In some embodiments, the weight ratio of the poly(oxyalkylene) block copolymer to the weight ratio of the at least one non-ionic surfactant is in a range from about 1%-99% to about 99%-1%. Specifically, the weight ratio of the poly(oxyalkylene) block copolymer to the weight ratio of the at least one non-ionic surfactant is from about 1% to 99%; alternatively, the weight ratio of the poly(oxyalkylene) block copolymer to the weight ratio of the at least one surfactant is about 50% to 50%; alternatively, the weight ratio of the poly(oxyalkylene) block copolymer to the weight ratio of the at least one surfactant is about 99% to 1%.

In some embodiments, the weight ratio of the mixture of at least two poly(oxyalkylene) block copolymers is 1%-99% relative to the weight of the bone repair composition. This weight ratio may be from 1-10%, 10-20%, 20-30%, 30%-40%, 40%-50%, 50%-60%, 60%-70%, 70%-80%, 80%-90%, or 90-99%. Alternatively, this weight ratio may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. The material may have the consistency of a solid, gel, putty, or any other non-liquid substance at room temperature.

In some embodiments, where the bone repair composition comprises two poly(oxyalkylene) block copolymers, the weight ratio of a first poly(oxyalkylene) block copolymer to the weight ratio of a second poly(oxyalkylene) block copolymer is in the range of from about 1%-99% to about 99%-1%. Specifically, the weight ratio of a first poly(oxyalkylene) block copolymer to the weight ratio of a second poly(oxyalkylene) block copolymer is about 1% to 99%; alternatively, the weight ratio of a first poly(oxyalkylene) block copolymer to the weight ratio of a second poly(oxyalkylene) block copolymer is about 50% to 50%; and alternatively, the weight ratio of a first poly(oxyalkylene) block copolymer to the weight ratio of a second poly(oxyalkylene) block copolymer is about 99% to 1%. The compositions may vary in molecular weight and be blended in ratios of 10:1 to 1:10.

The compositions may further comprise ions and other compounds that may be dissolved in water. For example, the addition of salts, such as PBS, can enhance solidification and setting properties of poloxamers. Divalent salts may be particularly useful to improve the rheological properties of compositions containing poloxamer mixtures and bioactive glass materials as well as those of compositions containing poloxamers and other solid bone repair materials.

The biocompatible or bioactive bone repair material may be osteoinductive, osteoconductive, or a material that is both osteoinductive and osteoconductive. The bone repair material may be xenogeneic, allogeneic, autogenic, and/or alloplastic.

In certain embodiments, the biocompatible or bioactive bone repair material may also be any combination of various therapeutic materials.

In certain embodiments, the composition may be prepared as a composite with a biocompatible or bioactive agent, such as a bioactive glass ceramic which contains silica or boron. The ceramic releases calcium and silicate or calcium and boron ions, which facilitate the differentiation and proliferation of osteoblasts (defined as osteostimulation), which in turn increases the rate of regeneration of hard tissue.

In addition, the bioactive glass component undergoes an ion exchange with the surrounding body fluid to form hydroxyapatite analogous to bone mineral. More specifically, dissolution of the bioactive glass ceramics releases the calcium and silicate or calcium and boron ions, which stimulate the genes responsible of the differentiation and proliferation of osteoblast cells within the bony defect upon implantation. It is believed that this genetic response is activated through the introduction of the genetic cascade responsible for the osteoblast proliferation and subsequently promotes the increased rate of regeneration of hard tissue.

In certain embodiments, the bone repair material is bioactive glass. Bioactive glass may be melt-derived or sol-gel derived. Depending on their composition, bioactive glasses may bind to soft tissues, hard tissues, or both soft and hard tissues. The composition of the bioactive glass may be adjusted to modulate the degree of bioactivity. Furthermore, borate may be added to bioactive glass to control the rate of degradation.

In some embodiments, the bioactive glass contains silica and/or boron as well as other ions such as sodium and calcium.

Certain embodiments relate to an irrigation resistant bone repair composition that includes a biocompatible or bioactive bone repair material suspended in a mixture of at least two non-random poly(oxyalkylene) block copolymers.

Certain further embodiments relate to an irrigation resistant bone repair composition that further includes at least one element selected from the group consisting of Li, Na, K, Mg, Sr, Ti, Zr, Fe, Co, Cu, Zn, Al, Ga, P, N, S, F, Cl, and I. For example, small amounts of iodine, fluorine or silver can provide antimicrobial properties, while small amount of copper can promote angiogenesis (i.e., aid in the formation of blood vessels).

The preferred embodiment includes non-random ethylene oxide and propylene oxide block copolymers as carriers for melt and sol-gel derived bioactive glasses. The composites range from 1 to 99% of a mixture of non-random EOPO block copolymers which is conversely 1-99% bioactive glass. The compositions may vary in molecular weight and may be blended in ratios of 10:1 up to 1:10. The composition, porosity and particle sizes of the bioactive glass may vary. Compositions of the glass may comprise from 0-90% silica or 0-90% boric acid with a plurality of other elements including Li, Na, K, Mg, Sr, Ti, Zr, Fe, Co, Cu, Zn, Al, Ga, P, N, S, F, Cl, and I. The particles of the glass may range in size from 0.01 μm to 5 mm. The embodiments take the consistency of a gel, putty, or waxy solid at room temperature.

In certain embodiments, bioactive glass is in the form of a particle. The composition, porosity and particle sizes of the bioactive glass may vary. In certain preferred embodiments, the particles of the glass may range in size from 0.01 μm to 5 mm. In certain embodiments, the bioactive glass comprises 0-80% 1000-2000 um bioactive glass, 0-90% 90-710 um bioactive glass, and 0-90% 32-125 um bioactive glass.

Exemplary compositions are provided in Table 1 below:

TABLE 1

| | | 90- | 32-125 | | % Glass |
|---|---|---|---|---|---|
| Sample | 1-2 mm | 710 μm | μm | Carrier | loading |
| 1 | 20% | 20% | 30% | 50% Poloxamer 407 | 65% |
| | | | | 50% Poloxamer 124 | |
| 2 | 20% | 20% | 30% | 60% Poloxamer 124 | 66.70% |
| | | | | 40% Poloxamer 407 | |
| 3 | 20% | 20% | 30% | 60% Poloxamer 124 | 71% |
| | | | | 40% Poloxamer 407 | |
| 4 | 20% | 20% | 30% | 50% Poloxamer 407 | 74% |
| | | | | 50% Poloxamer 124 | |
| 5 | 20% | 20% | 30% | 40% Poloxamer 124 | 65% |
| | | | | 60% Poloxamer 407 | |
| 6 | 35% | 35% | 30% | 40% Poloxamer 124 | 65.80% |
| | | | | 60% Poloxamer 407 | |
| 7 | 35% | 35% | 30% | 50% Poloxamer 407 | 71% |
| | | | | 50% Poloxamer 124 | |
| 8 | 35% | 35% | 30% | 60% Poloxamer 124 | 71% |
| | | | | 40% Poloxamer 407 | |
| 9 | 50% | 25% | 25% | 40% Poloxamer 124 | 65% |
| | | | | 60% Poloxamer 407 | |
| 10 | 50% | 25% | 25% | 50% Poloxamer 407 | 70.40% |
| | | | | 50% Poloxamer 124 | |
| 11 | 47.60% | 38.10% | 14.30% | 40% Poloxamer 124 | 66.70% |
| | | | | 60% Poloxamer 407 | |
| 12 | 47.60% | 38.10% | 14.30% | 50% Poloxamer 407 | 71% |
| | | | | 50% Poloxamer 124 | |
| 13 | 55% | 30% | 15% | 40% Poloxamer 124 | 68% |
| | | | | 60% Poloxamer 407 | |
| 14 | 35% | 35% | 30% | 40% Poloxamer 124 | 72.40% |
| | | | | 60% Poloxamer 407 | |
| 15 | 0% | 81.16% | 18.84% | 40% Poloxamer 124 | 69% |
| | | | | 60% Poloxamer 407 | |
| 16 | 45% | 40% | 15% | 40% Poloxamer 124 | 73% |
| | | | | 60% Poloxamer 407 | |
| 17 | 45% | 40% | 15% | 40% Poloxamer 124 | 70% |
| | | | | 60% Poloxamer 407 | |
| 18 | 45% | 40% | 15% | 40% Poloxamer 124 | 67% |
| | | | | 60% Poloxamer 407 | |
| 19 | 45% | 40% | 15% | 40% Poloxamer 124 | 60% |
| | | | | 60% Poloxamer 407 | |

Additional compositions are provided in Tables 2 and 3 below:

TABLE 2
Substituted Poloxamer 124
RK-01-78

| | 1-2 | 90- | 32- | | | | % | | |
|---|---|---|---|---|---|---|---|---|---|
| Sample | mm | 710 | 125 | Substitute | Pol 407 | HA | BG | % carrier | |
| 20 | 30.15 | 26.8 | 10.05 | 13.2 | 19.8 | 0 | 67 | 13.2 | Polyethylene Glycol 400 |
| 21 | 30.15 | 26.8 | 10.05 | 13.2 | 19.8 | 0 | 67 | 13.2 | Dipropylene Glycol |
| 22 | 30.15 | 26.8 | 10.05 | 13.2 | 19.8 | 0 | 67 | 13.2 | Lecithin |

TABLE 3
RK-01-78-B with Higher % BG and +HA
JB-01-16

| | 1-2 | 90- | 32- | | | | % | % |
|---|---|---|---|---|---|---|---|---|
| Sample | mm | 710 | 125 | PEG 400 | Pol 407 | HA | BG | Pol |
| 1A | 30.15 | 26.8 | 10.05 | 13.2 | 19.8 | 0.3 | 67 | 33 |
| 1B | 25.15 | 30.8 | 14.05 | 11.7 | 18.3 | 0.3 | 70 | 30 |
| 1C | 11.55 | 42.35 | 23.1 | 11.5 | 11.5 | 0.3 | 77 | 23 |

Further exemplary compositions are provided in Tables 4, 5 and 6 below:

TABLE 4
Subbed Pol 407; Std Formulation
JB-01-24

| | 1-2 | 90- | 32- | Carrier | | % | | % |
|---|---|---|---|---|---|---|---|---|
| Sample | mm | 710 | 125 | 1 | Pol 124 | BG | Condition | HA |
| 1A | 30.15 | 26.8 | 10.05 | 19.8 | 13.2 | 67% | Carrier 1 = PEG 400 Distearate | 0 |
| 2A | 30.15 | 26.8 | 10.05 | 19.8 | 13.2 | 67% | Carrier 1 = Sorbitan Tristearate | 0 |
| 3A | 30.15 | 26.8 | 10.05 | 19.8 | 13.2 | 67% | Carrier 1 = PEG 2000 Monomethyl Ether | 0 |
| 1B | 30.15 | 26.8 | 10.05 | 19.8 | 13.2 | 67% | Carrier 1 = PEG 400 Distearate | 0.3 |
| 2B | 30.15 | 26.8 | 10.05 | 19.8 | 13.2 | 67% | Carrier 1 = Sorbitan Tristearate | 0.3 |
| 3B | 30.15 | 26.8 | 10.05 | 19.8 | 13.2 | 67% | Carrier 1 = PEG 2000 Monomethyl Ether | 0.3 |

TABLE 5
Subbed Pol 124; Std Formulation
JB-01-22

| | 1-2 | 90- | 32- | | Carrier | % | | % |
|---|---|---|---|---|---|---|---|---|
| Sample | mm | 710 | 125 | Pol 407 | 2 | BG | Condition | HA |
| 1A | 30.15 | 26.8 | 10.05 | 19.8 | 13.2 | 67% | Carrier 2 = Polysorbate 20 | 0 |
| 2A | 30.15 | 26.8 | 10.05 | 19.8 | 13.2 | 67% | Carrier 2 = Polysorbate 80 | 0 |
| 3A | 30.15 | 26.8 | 10.05 | 19.8 | 13.2 | 67% | Carrier 2 = PEG 400 Monostearate | 0 |
| 4A | 30.15 | 26.8 | 10.05 | 19.8 | 13.2 | 67% | Carrier 2 = PEG 400 Distearate | 0 |
| 1B | 30.15 | 26.8 | 10.05 | 19.8 | 13.2 | 67% | Carrier 2 = Polysorbate 20 | 0.3 |
| 2B | 30.15 | 26.8 | 10.05 | 19.8 | 13.2 | 67% | Carrier 2 = Polysorbate 80 | 0.3 |
| 3B | 30.15 | 26.8 | 10.05 | 19.8 | 13.2 | 67% | Carrier 2 = PEG 400 Monostearate | 0.3 |
| 4B | 30.15 | 26.8 | 10.05 | 19.8 | 13.2 | 67% | Carrier 2 = PEG 400 Distearate | 0.3 |

TABLE 6
Substituted Poloxamer 124
RK-01-78

| | 1-2 | 90- | 32- | | | | % | % | |
|---|---|---|---|---|---|---|---|---|---|
| Sample | mm | 710 | 125 | Substitute | Pol 407 | HA | BG | Pol | Sub for Pol 124 |
| A | 30.15 | 26.8 | 10.05 | 13.2 | 19.8 | 0 | 67 | 33 | Propylene Glycol |
| B | 30.15 | 26.8 | 10.05 | 13.2 | 19.8 | 0 | 67 | 33 | Polyethylene Glycol 400 |
| C | 30.15 | 26.8 | 10.05 | 13.2 | 19.8 | 0 | 67 | 33 | Glycerol |
| D | 30.15 | 26.8 | 10.05 | 13.2 | 19.8 | 0 | 67 | 33 | Dipropylene Glycol |
| E | 30.15 | 26.8 | 10.05 | 13.2 | 19.8 | 0 | 67 | 33 | Lecithin |
| F | 30.15 | 26.8 | 10.05 | 13.2 | 19.8 | 0 | 67 | 33 | MCT Oil |

The various types of bioactive glass that may be used as bone repair material were previously described In U.S. Pub. No. US 2014/0079789, entire content of which is incorporated herein by reference.

Specifically, the bioactive glass material may have silica, sodium, calcium, strontium, phosphorous, and boron present, as well as combinations thereof. In some embodiments, sodium, boron, strontium, and calcium may each be present in the compositions in an amount of about 1% to about 99%, based on the weight of the bioactive glass. In further embodiments, sodium, boron, strontium and calcium may each be present in the composition in about 1%, about 2%, about 3%, about 4%, about 5%, about 6%, about 7%, about 8%, about 9%, or about 10%. In certain embodiments, silica, sodium, boron, and calcium may each be present in the composition in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, strontium, and boron with only traces of the other(s). The term “about” as it relates to the amount of calcium phosphate present in the composition means +/−0.5%. Thus, about 5% means 5+/−0.5%.

The bioactive glass materials may further comprise one or more of a silicate, borosilicate, borate, strontium, or calcium, including SrO, CaO, P₂O₅, SiO₂, and B₂O₃. In certain embodiments, bioactive glass includes about 15-45% CaO, about 30-70% SiO₂, about 0-25% Na₂O, about 0-17% P₂O₅, about 0-10% MgO and about 0-5% CaF₂.

An exemplary bioactive glass is 45S5, which includes 46.1 mol % SiO₂, 26.9 mol % CaO, 24.4 mol % Na₂O and 2.5 mol % P₂O₅.

An exemplary borate bioactive glass is 45S5B1, in which the SiO₂ of 45S5 bioactive glass is replaced by B₂O₃.

Other exemplary bioactive glasses include 58S, which includes 60 mol % SiO₂, 36 mol % CaO and 4 mol % P₂O₅, and S70C30, which includes 70 mol % SiO₂ and 30 mol % CaO.

In any of these or other bioactive glass materials of the invention, SrO may be substituted for CaO.

The following composition provided in Table 4 below, having a weight % of each element in oxide form in the range indicated, will provide one of several bioactive glass compositions that may be used to form a bioactive glass material:

TABLE 7

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-5 |
| CaF | 0-35 |

The bioactive glass can be in the form of a three-dimensional compressible body of loose glass-based fibers in which the fibers comprise one or more glass-formers selected from the group consisting of P₂O₅, SiO₂, and B₂O₃. Some of the fibers have a diameter between about 100 nm and about 10,000 nm, and a length:width aspect ratio of at least about 10. The pH of the bioactive glass can be adjusted as-needed.

The bioactive glass particles, fibers, meshes or sheets may further comprise any one or more of adhesives, grafted bone tissue, in vitro-generated bone tissue, collagen, calcium phosphate, stabilizers, antibiotics, antibacterial agents, antimicrobials, drugs, pigments, X-ray contrast media, fillers, and other materials that facilitate grafting of bioactive glass to bone.

The silica and/or calcium ions released by the bioactive glass may improve the expression of osteostimulative genes. The silica and/or calcium ions may also increase the amount of and efficacy of proteins associated with such osteostimulative genes. In several embodiments, the bone repair material is osteostimulative and can bring about critical ion concentrations for the repair and regeneration of hard tissue without the necessity of any therapeutic materials or agents.

In some embodiments, the bone repair material is 45S5 bioactive glass. The 45S5 bioactive glass may vary in size from 1 micrometer to 5 millimeters. The bioactive glass may be about 1-5 micrometers, about 5-15 micrometers, about 15-50 micrometers, about 50-200 micrometers, about 200-1,000 micrometers, about 1-2 millimeters, about 2-3 millimeters, about 3-4 millimeters, or about 4-5 millimeters.

In some embodiments, the bioactive glass particle has a diameter of between about 1 micrometer and about 2,000 micrometers.

In some embodiments, the bone repair material is a composition comprising calcium salt and silica. The silica is in the form of an inorganic silicate that is adsorbed onto the surface of the calcium salt. The silica is not incorporated into the structure of the calcium salt. The composition may be bioactive. These and other bone repair materials are described in U.S. Patent Pub. No. US 2013/0330410, the entire content of which is herein incorporated by reference.

In some embodiments, the bone repair material is a composition comprising suspended autograft bone particles and suspended bioactive glass particles. Similar bone repair materials are described in U.S. Provisional Patent Application No. 61/641,961, filed on May 3, 2012, the entire content of which is incorporated herein by reference, and in U.S. Provisional Patent Application No. 61/623,357, filed on Apr. 12, 2012, the entire content of which is herein incorporated by reference.

The suspended bioactive glass particle may comprise SiO₂. Alternatively, the suspended bioactive glass particle may comprise P₂O₅, PO₃. or PO₄. The suspended bioactive glass particle may comprise B₂O₃ as well. In some embodiments, the suspended bioactive glass particle may comprise 40-60% SiO₂, 10-20% CaO, 0-4% P₂O₅, and 19-30% NaO. The suspended bioactive glass particle may further comprise a carrier selected from the group consisting of hydroxyapatite and tricalcium phosphate.

The bioactive glass particle may be pretreated in a solution comprising one or more of blood, bone marrow aspirate, bone-morphogenetic proteins, platelet-rich plasma, and osteogenic proteins.

In various embodiments, the bioactive glass particle may not include any substantial amount of polymer.

In some embodiments, the bone repair material may be bioactive glass coated with a glycosaminoglycan, in which the glycosaminoglycan is bound to the bioactive glass. This and other bone repair materials are described in U.S. Patent Pub. No. US 2014/0079789, the entire content of which is incorporated by reference herein. The glycosaminoglycan may be bound to the bioactive glass by means of an ionic bond or a covalent bond. The glycosaminoglycan may be heparin, heparan sulfate, chondroitin sulfate, dermatan sulfate, keratan sulfate, or hyaluronic acid.

In certain other embodiments, the bone repair material may include surface immobilized peptides, as previously described in U.S. Provisional Application No. 61/974,818, filed on Apr. 3, 2014, which is incorporated herein in its entirety.

In some further embodiments, the bone repair material is a bimodal bioactive glass composition comprising large bioactive glass particles and small bioactive glass particles. The large bioactive glass particles have a substantially spherical shape and a mean diameter of between about 90 micrometers and about 2,000 micrometers. The small bioactive glass particles have a substantially spherical shape and a mean diameter of between about 10 micrometers and about 500 micrometers.

In some embodiments, the bone repair material is a trimodal bioactive glass composition comprising large bioactive glass particles, medium bioactive glass particles, and small bioactive glass particles. The large bioactive glass particles have a substantially spherical shape and a mean diameter of between about 500 micrometers and about 5,000 micrometers. The medium bioactive glass particles have a substantially spherical shape and a mean diameter of between about 90 micrometers and about 710 micrometers. The small bioactive glass particles have a substantially spherical shape and a mean diameter of between about 1 micrometers and about 125 micrometers.

In any of the above embodiments, small bioactive glass fibers may be added to the bone repair material. The small bioactive glass fibers have a diameter of less than 2 millimeters. The small bioactive glass fibers may be present in up to 40% by weight relative to the total weight of the bioactive glass. In various embodiments, the weight ratio of small bioactive glass fibers to total weight of the bioactive glass may be from 0-10%, 0-5%, 5-10%, 5-15%, 10-15%, 10-20%, 15-20%, 15-25%, 20-25%, 20-30%, 25-30%, 25-35%, 30-35%, 30-40%, or 35-40%. The weight ratio of small bioactive glass fibers to total weight of the bioactive glass may be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, any subset of the bioactive glass present, such as bioactive glass particles and/or small bioactive glass fibers, may be coated with silane as described in Verne et al. (Verne et al., “Surface functionalization of bioactive glasses,” J. Biomed. Mater. Res. A., 90(4):981-92 (2009)). The silane or other functional coatings may then allow for binding of proteins onto the bioactive glass, such as BMP-2.

In some embodiments, any subset of the bioactive glass present, such as bioactive glass particles and/or small bioactive glass fibers, may have additional silicate chains present on them. The additional silicate chains may allow the bioactive glass particles and fibers to interact with one another, as well as with the EO and PO groups on the poloxamers. The effect of these interactions may be to reduce the surface area of the filler, increase resin demand, and to allow for higher filler loadings.

In some embodiments, any subset of the bioactive glass present, such as bioactive glass particles and/or small bioactive glass fibers, may have added hydroxyl triethoxysilanes coated onto the glass. Some of these silanes are available from Gelest, Inc. For example, the glass may be coated with hydroxyl(polyethyleneoxy) propyltriethoxysilane. Additionally, the glass may be coated with other organic substituted ethoxy- and methoxy-silanes that are effective to create an interaction between the coated glass and the EO/PO carrier.

In any of the above embodiments, the irrigation resistant bone repair composition may be applied by a syringe at ambient temperature. After application to the bone or other site within the body at 37° C., the bone repair composition will harden and have a substantially lower tendency to migrate away from the application site.

More viscous bone repair compositions may be applied by painting the composition onto a site at or near the bone defect. Alternatively, more viscous bone repair compositions may be extruded onto the site in the form of a bead.

Certain embodiments relate to a method for treating hard tissues, such as bones using the irrigation resistant bone repair composition.

Certain other embodiments relate to a method for treating a bone having a bone defect comprising contacting the bone at or near the site of the bone defect with the irrigation resistant bone repair composition of any of the above-described embodiments.

Any of the above-described materials or methods may be undertaken to treat any number of bone defects. As such, certain further embodiments relate to a method for treating a bone having a bone defect comprising placing an irrigation resistant bone repair composition of any one of the above-described embodiments at a site of a bone gap or a bone defect.

A bone defect may include bony structural disruptions, in which repair is needed or may be a gap in the bone or may arise from lack of adequate bone regeneration. A bone defect may be a void, which is understood to be a three-dimension defect that includes a gap, cavity, hole or other substantial disruption of the structural integrity of the bone or joint. The bone defects may also be fractures. The bone defects may also arise in the context of oral bone defects. The different types of bone defects are apparent to those of ordinary skill in the art. Gaps may be at least 2.5 cm and are generally in the range of 3-4 cm. This size is large enough so that spontaneous repair is not likely to occur and/or be complete. Exemplary bone defects include tumor resection, fresh fractures, cranial and facial abnormalities, spinal fusions, and loss of bone from the pelvis.

The various embodiments of the invention may be particularly useful with respect to orthopedic and spine processes because the material will stabilize and hold a better structure as it becomes more solidified when it heats up to body temperature.

Certain further embodiments relate to a method for treating a bone having a bone defect comprising placing an irrigation resistant bone repair composition of any one of the above-described embodiments at a bone gap or a bone defect.

In some embodiments, any of the above-described materials or methods may be combined with autograft bone chips for placement onto or near a bone defect. The materials may be a liquid or a gel at room temperature with the autograft bone chips suspended therein. Upon placement at or near the bone defect, the material will solidify around the autograft bone chips and serve to prevent the autograft bone chips from migrating away from the surgical sites.

In some embodiments, any of the above-described materials or methods may be combined with particles containing allogeneic or xenogeneic bone mineral for placement onto or near a bone defect. The materials may be a liquid or a gel at room temperature with the particles suspended therein. Upon placement at a surgical site, which is at or near the bone defect, the material will solidify around the particles and serve to prevent the particles from migrating away from the surgical site.

In various embodiments of the invention, the bone repair material is entirely synthetic. Advantages of using such a bone repair material include the elimination of substantially all risk of disease transmission.

In various embodiments of the invention, the bone repair material is not a natural bone material or a synthetic bone material.

Further embodiments relate to kits that include an irrigation resistant bone repair composition including a biocompatible or bioactive bone repair material, and a mixture of at least one non-random poly(oxyalkylene) block copolymer and at least one surfactant other than the non-random poly(oxyalkylene) block copolymer. The non-ionic surfactant or similar material other than the non-random poly(oxyalkylene) block copolymer is selected from the group consisting of fatty Alcohols (e.g., stearyl alcohol), alkoxylated alcohols (e.g., Ecosurf LF 45), alkoxylated alkylphenols (e.g., Triton X-100), alkoxylated fatty amides (e.g., polyethoxylated tallow amine), alkoxylated fatty esters (e.g., PEG 400 Monostearate), alkoxylated fatty ethers (e.g., polyethylene glycol lauryl ether (Brij L23), alkoxylated sorbitan esters (e.g., Span 85 (sorbitan trioleate)), alkoxylated sorbitan esters (e.g., Polysorbate 20 and PolySorbate 80 also referred to as Tween 20 and Tween 80), fatty acid esters or polyol esters (e.g., glycerol monostearate, PEG coconut triglycerides), polyalkylene glycols (e.g., PEG 400 and PEG 600). Specific examples of surfactants other than the non-random poly(oxyalkylene) block copolymer include sorbitan tristearate, polysorbate 20, polysorbate 80, Polyoxyethylene 7 Coconut, Glycerides, PEG 400 Monostearate, PEG 2000 Monomethylether, and PEG 400 Distearate. At least one of the surfactants in the composition has a melting point above room temperature, and more preferably above body temperature. Other suitable surfactant materials may be used.

Further embodiments relate to kits that include an irrigation resistant bone repair composition including a biocompatible or bioactive bone repair material, and a mixture of at least two non-random poly(oxyalkylene) block copolymers.

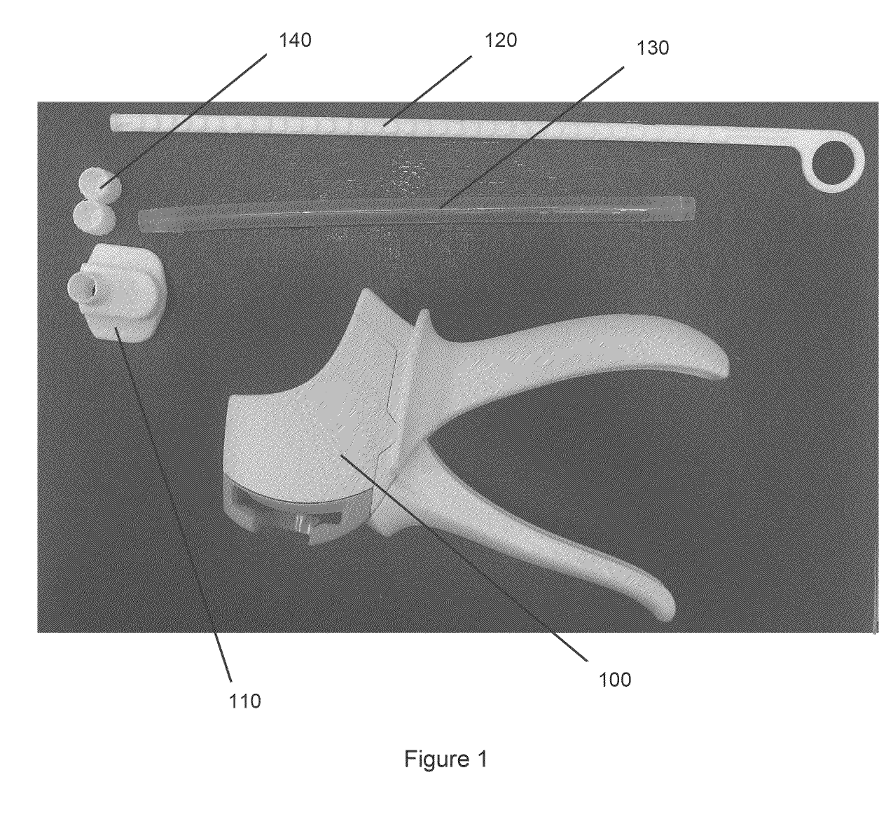

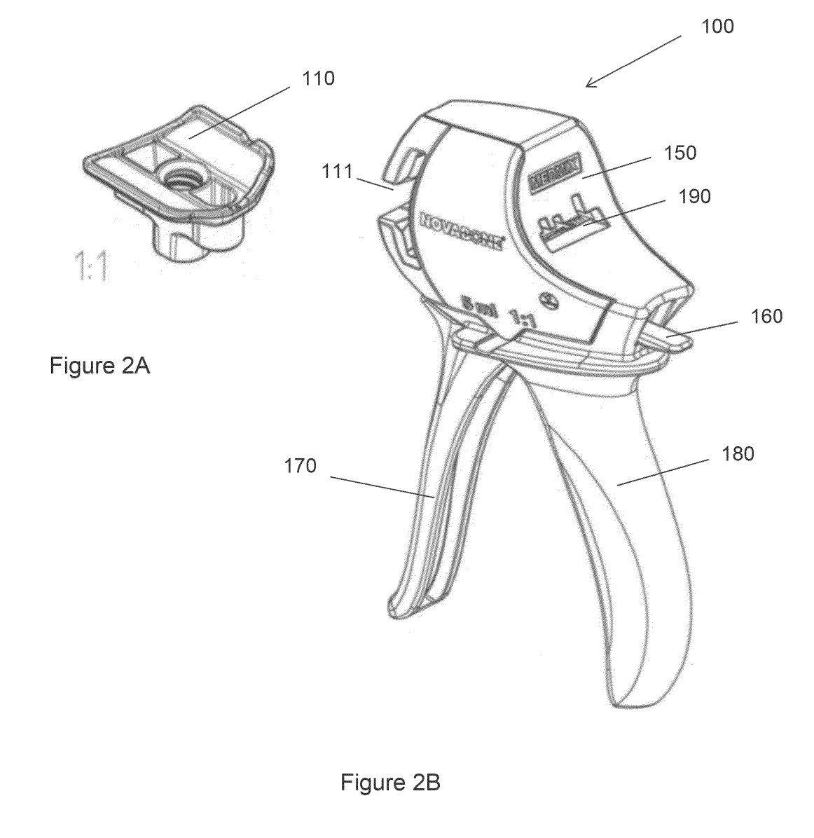

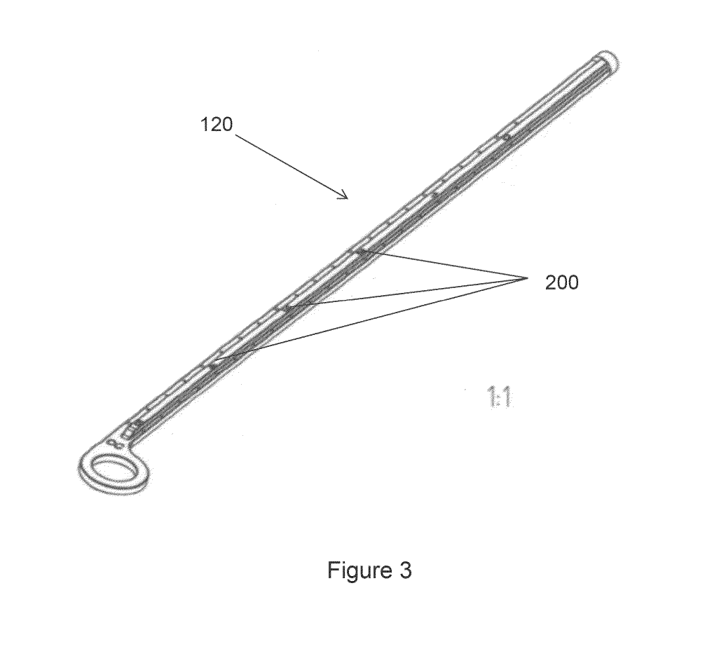

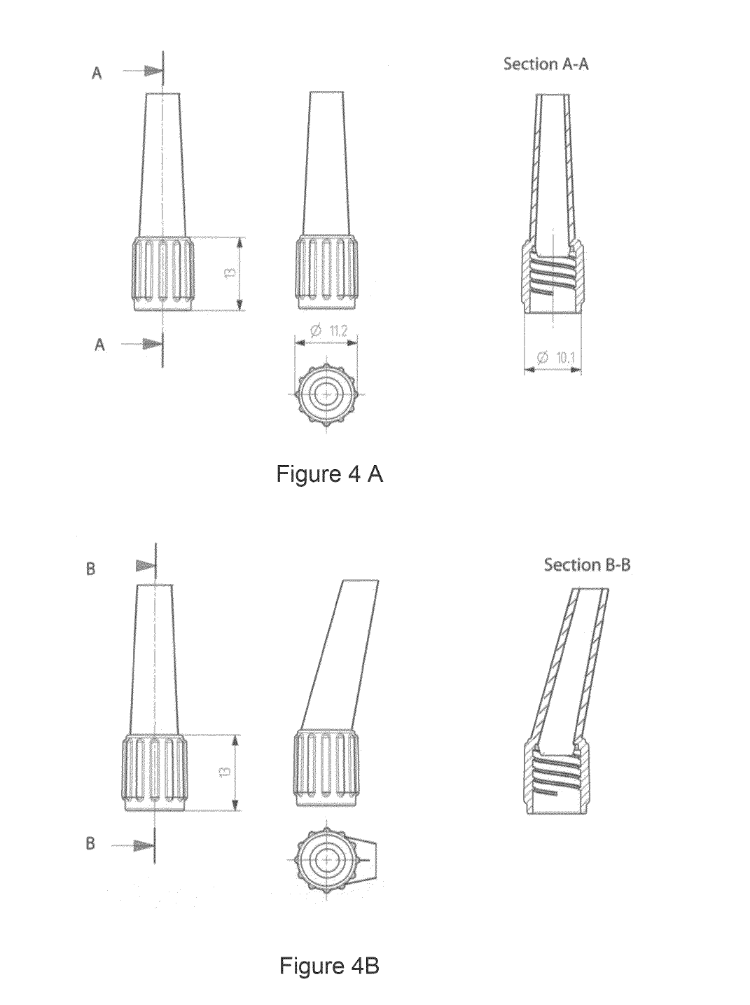

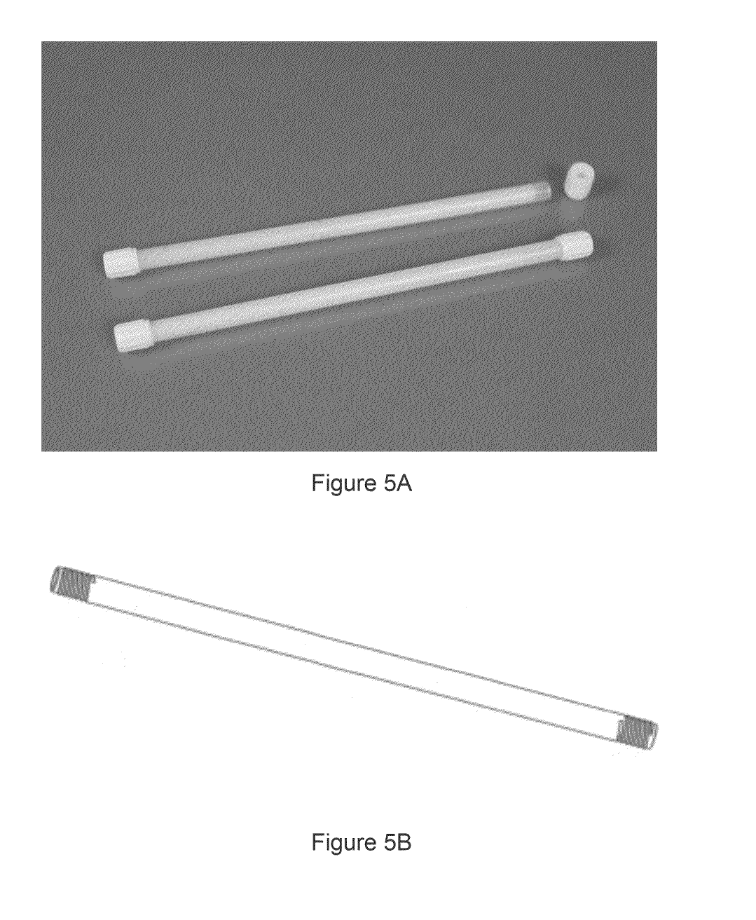

The kits may further include a dispensing gun, syringe, clam shell, or other suitable delivery device and accompanying accessories. Specifically, referring to FIGS. 1 and 2A-B, the exemplary dispensing gun 100, adapter 110, plunger 120 (see also FIG. 3), tube(s) 130 (see also FIGS. 5A and 5B), caps 140, and assorted dispensing tips (optional; FIG. 4A and FIG. 4B) that may be included with the kits are shown. The irrigation resistant bone repair composition may be deposited into the tube(s) 130 as part of the kit (FIG. 5A). An exemplary kit for delivery of other materials, such as Bioactive Synthetic Bone Graft Putty is currently being sold by NOVABONE® (NOVABONE® Bioactive Synthetic Bone Graft Putty MIS Cartridge Delivery System, NovaBone Products, LLC, Alachua, Fla.).

Referring to FIGS. 2A-B, the dispensing gun 100 may include a cover 150, a latch 160, a lever 170 and a handle 180 (FIG. 2B). The adapter 110 (shown also in FIG. 2A) may be inserted into the dispensing gun at an opening 111. A plunger (not shown) may be inserted through the front of the gun and pushed through the opening in the back 190 of the gun.

FIG. 3 depicts an exemplary plunger 120 including gradient markings 200 facing up.

FIGS. 4A-B depict exemplary tips for use with the dispensing gun. The tips may be straight (FIG. 4A) or at an angle (FIG. 4B).

FIG. 5A is a picture of tubes filled with the irrigation resistant bone repair composition; FIG. 5B is a graphical illustration of an exemplary tube for use with the kit and specifically with the delivery gun described above. The tubes have a substantially constant inner diameter along their entire length such that the outlets have substantially the same inner diameters as the rest of the tubes.

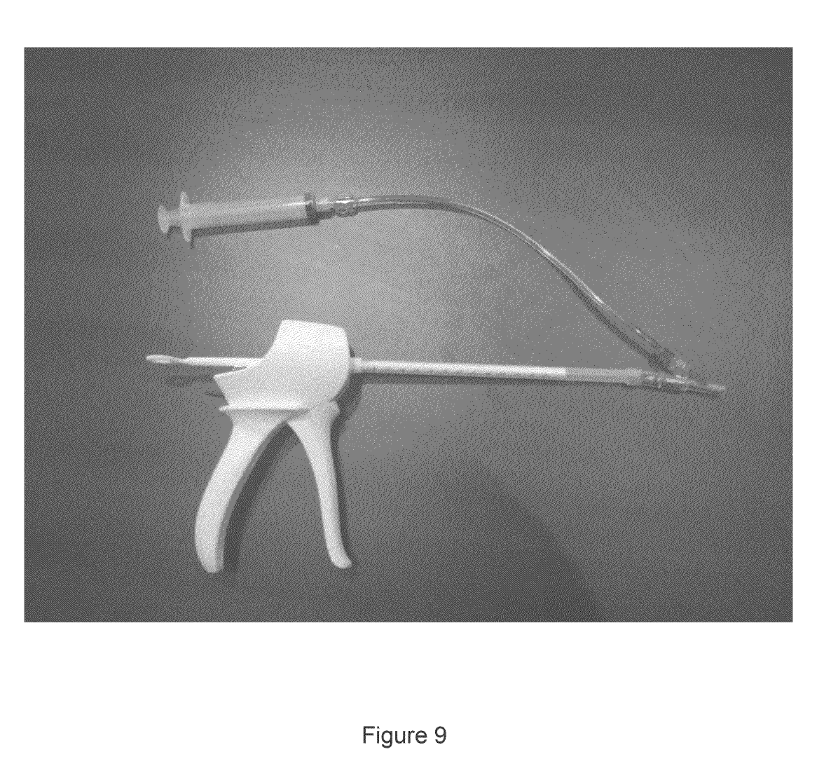

Optionally, a “Y” connector, luer syringe and a tube connector may be included to facilitate the simultaneous delivery of biologics and to maintain position during shipping (as shown in FIG. 9).

The components of a kit may be packaged and sold as a kit. The components of a kit may snap fit into a (inner) tray of a packaging and a retainer may be placed over the components of the kit to maintain position of the components during shipping. The inner tray may hold up to four tubes that can be prefilled with the irrigation resistant bone repair composition and capped on each end. The inner tray may also contain cavities for the placement of assorted tips, a “Y” connector, tube connector, a syringe and aspiration needle.

The inner tray may be sealed with a lid and placed into an outer tray also sealed with a lid. The sealed trays are radiation sterilized for use in medical applications. The sealed trays may then be placed in a box.

Immediately prior to use, the kit may be placed in an operating room and the outer tray is opened. The inner tray is removed by a sterile technician and placed into the sterile field.

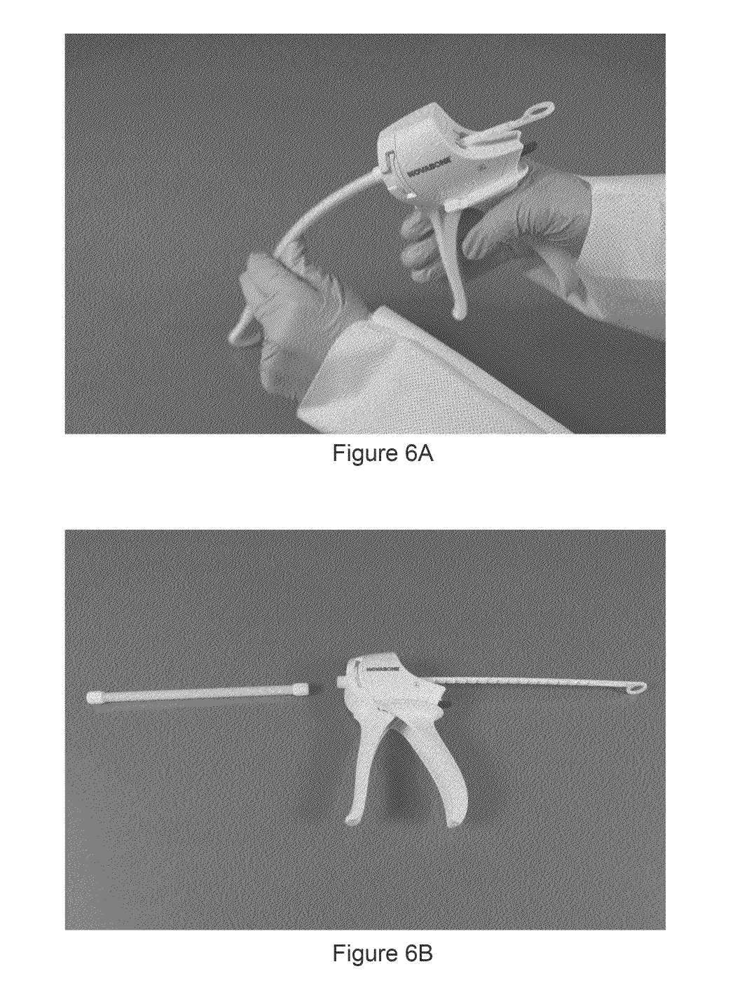

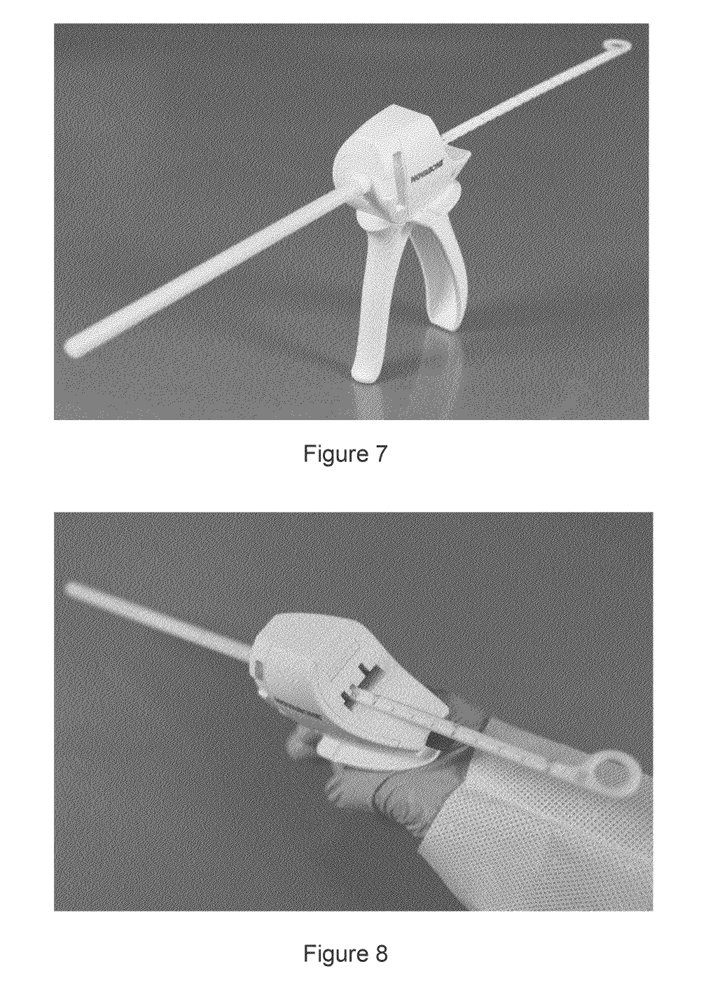

In the sterile field the inner tray is opened and the dispensing gun is assembled by inserting the finger grip of the plunger 120 (with the gradient markings 200 facing up and teeth facing down) through the opening in the front of the gun 100 and pushing the plunger through the back of the gun until the piston end of the plunger is seated completely within the gun (see FIGS. 6A, 7 and 8). The adapter 110 is then inserted into the front of the gun 100. Next a prefilled tube is removed from the inner tray. One cap is removed from the prefilled tube. The tube is threaded into the adapter and the other cap is removed from the tube (FIG. 6B). Optionally a tip can be placed on the end of the tube to direct the flow of the graft material.

The tip of the instrument may be placed into the surgical site. Upon pressing the trigger of the gun, the plunger is ratcheted forward to express the bone grafting material into the surgical site. The dispensing gun consists of, a handle, in which a block is moved forward through pressing the trigger which engages the teeth of the plunger moving the piston forward displacing the material from the tube. The trigger is manually disengaged by pushing the lever at the back of the dispensing gun upward allowing the plunger to be pulled back to a starting position. The first tube can be removed from the adapter and additional tubes can be threaded in place as needed.

Another embodiment involves altering the adapter for the attachment of two tubes and the plunger modified from a single piston to one have two pistons moving simultaneously with each compression of the trigger. Subsequently, the plungers dispense the material from the two tubes through a static mixer to facilitate the addition of a biological or drug material into the non-setting bone grafting material during injection into the surgical site. Any of the above-described aspects and embodiments of the invention may be in injectable form. Injection may occur by means of a syringe, for example. The compositions are particularly useful when injected in a gel or liquid form into a bone gap or bone defect. The injected gel or liquid would then solidify at body temperature when placed on or near the bone gap or the bone defect.

### Alternative Embodiments

Certain embodiments relate to an irrigation resistant bone repair composition comprising a biocompatible or bioactive bone repair material, and a mixture of at least two non-random poly(oxyalkylene) block copolymers. In the bone repair composition the poly(oxyalkylene) block copolymers are poloxamer polymers. In the composition, the poly(oxyalkylene) block copolymers are selected from the group consisting of poloxamer 407, poloxamer 124, poloxamers 188, poloxamer 237, and poloxamer 338. In the composition, the weight ratio of the mixture of at least two poly(oxyalkylene) block copolymers is 1%-99% relative to the weight of the bone repair composition. In the composition, the weight ratio of the mixture of at least two poly(oxyalkylene) block copolymers is 1%-20% relative to the weight of the bone repair composition. In the composition, the weight ratio of the mixture of at least two poly(oxyalkylene) block copolymers is 20%-30% relative to the weight of the bone repair composition. In the composition, the weight ratio of the mixture of at least two poly(oxyalkylene) block copolymers is 30%-40% relative to the weight of the bone repair composition. In the composition, the weight ratio of the mixture of at least two poly(oxyalkylene) block copolymers is 40%-50% relative to the weight of the bone repair composition. In the composition, the weight ratio of the mixture of at least two poly(oxyalkylene) block copolymers is 50%-60% relative to the weight of the bone repair composition. In the composition, the weight ratio of the mixture of at least two poly(oxyalkylene) block copolymers is 60%-70% relative to the weight of the bone repair composition. In the composition, the weight ratio of the mixture of at least two poly(oxyalkylene) block copolymers is 70%-80% relative to the weight of the bone repair composition. In the composition, the weight ratio of the mixture at least two poly(oxyalkylene) block copolymers is 80%-99% relative to the weight of the bone repair composition. In the composition, the bone repair composition comprises two poly(oxyalkylene) block copolymers, and the weight ratio of a first poly(oxyalkylene) block copolymer to the weight ratio of a second poly(oxyalkylene) block copolymer is about 1% to 99%. In the composition, the bone repair composition comprises two poly(oxyalkylene) block copolymers, and the weight ratio of a first poly(oxyalkylene) block copolymer to the weight ratio of a second poly(oxyalkylene) block copolymer is about 50% to 50%. In the composition, the bone repair composition comprises two poly(oxyalkylene) block copolymers, and the weight ratio of a first poly(oxyalkylene) block copolymer to the weight ratio of a second poly(oxyalkylene) block copolymer is about 99% to 1%. The composition is osteoconductive. The composition is osteostimulative. In the composition, the bone repair material is a bioactive glass or ceramic. In the composition, the bioactive glass is melt-derived bioactive glass or sol-gel derived bioactive glass. In the composition, the bioactive glass is in the form of a particle. In the composition, the bioactive glass particle comprises SiO₂. In the composition, the bioactive glass particle comprises P₂O₅, PO₃, or PO₄. In the composition, the bioactive glass particle comprises B₂O₃. In the composition, the bioactive glass particle comprises about 15-45% CaO, about 30-70% SiO₂, about 0-25% Na₂O, about 0-17% P₂O₅, about 0-10% MgO and about 0-5% CaF₂. Alternatively, in the composition, the bioactive glass particle comprises about 45% SiO₂, about 24.5% CaO, about 6% P₂O₅, and about 2.5% Na₂O. In the composition, the size of the bioactive glass particle is in a range from about 0.01 um to about 5 mm. In the composition, the bioactive glass comprises 0-80% 1000-2000 um bioactive glass, 0-90% 90-710 um bioactive glass, and 0-90% 32-125 um bioactive glass. In the composition, the bone repair material is one or more particles of bioactive glass coated with a glycosaminoglycan, wherein the glycosaminoglycan is bound to the bioactive glass. In the composition, the glycosaminoglycan is bound to the bioactive glass by means of an ionic bond. In the composition, the glycosaminoglycan is bound to the bioactive glass by means of a covalent bond. In the composition, the glycosaminoglycan is selected from the group consisting of heparin, heparan sulfate, chondroitin sulfate, dermatan sulfate, keratan sulfate, and hyaluronic acid. The bone repair composition further comprises at least one element selected from the group consisting of Li, K, Mg, Sr, Ti, Zr, Fe, Co, Cu, Zn, Al, Ag, Ga, P, N, S, F, Cl, and I. In the composition, the bioactive glass particle is pretreated in a solution comprising one or more of blood, bone marrow, bone marrow concentrate, bone-morphogenetic proteins, platelet-rich plasma, and osteogenic proteins. In the composition, the proteins used for pre-treatment are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof. The composition is in a form of a putty, paste, gel, or waxy solid. The composition, when implanted into a surgical site, maintains position and does not displace upon irrigation of the surgical site. The bone repair composition is for treating a bone defect or a bone gap. The bone repair composition is for treating a bone defect or a bone gap. The bone repair composition is for regeneration of hard tissues.

Certain other embodiments relate to an irrigation resistant putty or paste including the composition described directly above mixed with water, saline, blood, or BMA.

Certain further embodiments relate to a method for treating a bone having a bone gap or a bone defect comprising contacting the bone at or near the site of the bone defect with the bone repair composition described directly above.

Certain further embodiments relate to a kit comprising at least one tube comprising the bone repair composition described above, a dispensing gun, an adapter, and optionally, at least one dispensing tip. In the kit, the tube comprising the bone repair composition is capped. The kit further comprises a syringe. The kit further comprises at least one of “Y” connector, tube connector, and an aspiration needle.

### Example 1

### Poloxamers-Coated Bioactive Glass Particles for Bone Repair

The bone repair compositions were prepared by mixing two different poloxamers with bioglass particles as noted in Table 8 below.

TABLE 8

| | | | | | % | | | |
|---|---|---|---|---|---|---|---|---|
| Sample ID | 1-2 | 90-710 | 32-125 | | Glass | | | Post- |
| (notebook) | mm | μm | μm | Carrier | loading | Comments | Sterilization | sterilization |
| B | 20% | 20% | 30% | 50% Poloxamer 407 | 65% | | | Not sterile |
| | | | | 50% Poloxamer 124 | | | | |
| C | 20% | 20% | 30% | 60% Poloxamer 124 | 66.70% | | | Not applica- |
| | | | | 40% Poloxamer 407 | | | | ble |
| D | 20% | 20% | 30% | 60% Poloxamer 124 | 71% | handled better in water | | Not applica- |
| | | | | 40% Poloxamer 407 | | than C-not as wet. | | ble |
| | | | | | | More putty-like | | |
| E | 20% | 20% | 30% | 50% Poloxamer 407 | 74% | | | Not applica- |
| | | | | 50% Poloxamer 124 | | | | ble |
| F | 20% | 20% | 30% | 40% Poloxamer 124 | 65% | putty-like, compresses/ | | Not applica- |
| | | | | 60% Poloxamer 407 | | molds well | | ble |
| G | 35% | 35% | 30% | 40% Poloxamer 124 | 65.80% | | | Not applica- |
| | | | | 60% Poloxamer 407 | | | | ble |
| H | 35% | 35% | 30% | 50% Poloxamer 407 | 71% | | | Not applica- |
| | | | | 50% Poloxamer 124 | | | | ble |
| I | 35% | 35% | 30% | 60% Poloxamer 124 | 71% | | | Not applica- |
| | | | | 40% Poloxamer 407 | | | | ble |
| J | 50% | 25% | 25% | 40% Poloxamer 124 | 65% | density: 5 g = 2.9 cc | Sterilized | increased |
| | | | | 60% Poloxamer 407 | | | | handling |
| | | | | | | | | properties, |
| | | | | | | | | shed less |
| K | 50% | 25% | 25% | 50% Poloxamer 407 | 70.40% | | | Not applica- |
| | | | | 50% Poloxamer 124 | | | | ble |
| L | 47.60% | 38.10% | 14.30% | 40% Poloxamer 124 | 66.70% | | | Not applica- |
| | | | | 60% Poloxamer 407 | | | | ble |
| M | 47.60% | 38.10% | 14.30% | 50% Poloxamer 407 | 71% | | | Not applica- |
| | | | | 50% Poloxamer 124 | | | | ble |
| N | 55% | 30% | 15% | 40% Poloxamer 124 | 68% | density: 5 g = 2.8 cc | Sterilized | increased |
| | | | | 60% Poloxamer 407 | | | | handling |
| | | | | | | | | properties, |
| | | | | | | | | shed less |
| P | 35% | 35% | 30% | 40% Poloxamer 124 | 72.40% | lower 1-2 made the | Sterilized | increased |
| | | | | 60% Poloxamer 407 | | handling better, more | | handling |
| | | | | | | putty like. Glass not as | | properties, |
| | | | | | | obvious though | | shed less |
| S | 0% | 81.16% | 18.84% | 40% Poloxamer 124 | 69% | Easy to mold. Pre- | E-beam | Very good |
| | | | | 60% Poloxamer 407 | | pared by melting | sterilized | handling |
| | | | | | | poloxamers first then | | |
| | | | | | | combining dry materi- | | |
| | | | | | | als. Mixed with Thinky | | |
| | | | | | | mixer at 2000 rpm | | |
| T-73 | 45% | 40% | 15% | 40% Poloxamer 124 | 73% | Hardest to mold of T | E-beam | No signifi- |
| | | | | 60% Poloxamer 407 | | samples. Prepared by | sterilized | cant differ- |
| | | | | | | melting poloxamers | | ence in |
| | | | | | | together then adding | | handling |
| | | | | | | dry materials. Mixed in | | after sterili- |
| | | | | | | Thinky mixer | | zation |
| T-70 | 45% | 40% | 15% | 40% Poloxamer 124 | 70% | Better handling than | E-beam | No signifi- |
| | | | | 60% Poloxamer 407 | | 73% glass loading | sterilized | cant differ- |
| | | | | | | sample. Prepared as | | ence in |
| | | | | | | immediately above. | | handling |
| | | | | | | | | after sterili- |
| | | | | | | | | zation |
| T-67 | 45% | 40% | 15% | 40% Poloxamer 124 | 67% | Best handling of T | E-beam | No signifi- |
| | | | | 60% Poloxamer 407 | | samples. Easy to | sterilized | cant differ- |
| | | | | | | mold. | | ence in |
| | | | | | | Prepared as immedi- | | handling |
| | | | | | | ately above. | | after sterili- |
| | | | | | | | | zation. Per- |
| | | | | | | | | formed best. |
| | | | | | | | | Accelerated |
| | | | | | | | | aging and |
| | | | | | | | | real time |
| | | | | | | | | stability |
| | | | | | | | | testing was |
| | | | | | | | | performed |
| HA | 45% | 40% | 15% | 40% Poloxamer 124 | 60% | Prepared by melting | E-beam | Handling |
| | | | | 60% Poloxamer 407 | | poloxamers together, | sterilized | was most |
| | | | | | | adding dry materials, | | similar to S |
| | | | | | | and then allowing | | sample. |
| | | | | | | them to melt together | | Sample was |
| | | | | | | in 80° C. oven. Materi- | | also placed |
| | | | | | | als were mixed once | | on acceler- |
| | | | | | | melted and had a 0.5 | | ated aging |
| | | | | | | wt % of hyaluronic | | and real |
| | | | | | | acid added, then | | time stability |
| | | | | | | kneaded by hand as | | testing |
| | | | | | | they cooled to room | | |
| | | | | | | temperature. | | |

### Example 2

The purpose of the study was to compare the efficacy of a putty and irrigation resistant matrix prototypes in a rabbit femora condyle model. The materials were compared in critical-size defects in the rabbit distal femur model via qualitative and quantitative histologic analysis. The primary test period was 6 weeks.

Six (6) NZW rabbits underwent bilateral surgery to create a critical sized defect in the distal femoral condyle. In three (3) animals, one leg received one of the prototype devices (test article) and the contralateral side received the putty (control article). In the remaining three (3) animals, one leg received one of the prototype devices (test article) and the contralateral side received a different prototype device (test article]. Animals were sacrificed and necropsy was performed 39 days post-surgery. At sacrifice all grafted sites and regional lymph nodes were grossly examined for defect filling and reactivity. Remnant bone graft material and the regional lymph nodes were evaluated for any system toxicity via standard histologic methods.

Test and Control Articles and Schedule of Procedures:

Control Article: NovaBone putty

Test Article: Prototype A (EOPO putty)

Test Article: Prototype B (EOPO putty with HA)

Test Article: Prototype C (NovaBone putty with HA)

The schedule of procedures is outlined in the following Table 9:

| Event | Study Day |
|---|---|
| Test/Control Article | Day O |
| Administration | |
| Post Operative Care | Daily for at least ten (10) days following surgery. |
| Cageside Observations | Daily, including weekends and holidays after the |
| | completion of post operative care. |
| Body weight | Upon arrival, as a part of the quarantine release |
| | exam, within 24 hours prior to surgery, and prior |
| | to termination. |
| Necropsy | Day 39 |

Methods:

On the day of surgical procedures, the operative site and the area to be used for placement of the fentanyl patch were shaved immediately prior to surgery. Food was withheld between two (2) to six (6) hours prior to induction of anesthesia on the day surgical procedures were performed. Pre-operative vitals including color of mucous membranes, heart rate, respiratory rate, and body temperature were recorded in preparation for surgical procedures. Vital signs were also monitored at approximately 15-minute intervals during surgical procedures, and postoperatively at approximately 30-minute intervals for each animal. Animals were administered pre-anesthesia as described below and administered general anesthesia via face mask. Once transferred to the operating table the animals were positioned in dorsal recumbency and the abdomen was aseptically prepared and draped in sterile fashion.

Surgical Procedure:

Animals were selected for surgery in numerical order. Supplemental heat was not provided during surgical procedures due to the short duration. The surgical site was prepared by saturating with 70% isopropyl alcohol, washed with Povidone iodine surgical scrub, rinsed with sterile saline, washed again with Povidone iodine surgical scrub, rinsed with sterile saline, and coated with Topical Povidone iodine solution.

All animals underwent the same surgical procedure. A skin incision over the medial femoral condyle was made to expose the distallateral aspect of the femur. The periosteum was incised and a transverse surgical defect was created in the coronal plane using a manual drill with a series of sequentially larger drill bits increasing in diameter from 2 to 6 mm. The final diameter of the defect was 6 mm. The defect was approximately 10 mm in depth extending from the lateral cortex to the medial cortical wall. Saline irrigation was applied as necessary to remove any debris from the site.

After creating the defect a final rinse of saline was applied to remove any residual particulate matter and gauze was inserted to dry the bony defect. The gauze was removed and the site was implanted with approximately 0.3 to 0.4 cc of the appropriate graft material. Caution was used to avoid excessive compression during insertion of the material into the defect. Care was used to ensure adequate contact between the implant and the medial and proximal margins of cut bone.

Multi-layer suturing was performed on the joint capsule, internal musculature, and skin using non-absorbable sutures (3.0 Ethilon with FS-1 needle) to eliminate the potential confounding effects of resorbable suture materials.

Post-Operative Care:

(a) Intensive Care Monitoring:

A bolus of 20 mL of lactated ringers was provided subcutaneously postoperatively. Post operative monitoring was performed by monitoring the vital signs at approximate 30-minute intervals. Animals were placed on heating pads to help increase body temperature as the animal recovered from anesthesia. Animals were placed back in their home cages once they attained sternal recumbence. Animals were not removed from their cages until Day 3 out of an abundance of caution in order to minimize stress to both the surgical site and to the animal itself during the initial recovery period.

(b) Post-Operative Daily Observations of General Health:

Animals were observed daily for at least ten (10) days following surgical procedures. In addition, rabbits were closely monitored for pain, neurologic complications, and ambulatory function. After ten (10) days animal observations were performed as described below.

(c) Surgical Incision Site Observation:

The surgical incision site was observed for wound healing and signs of infection daily for at least ten (10) days following surgery. The incision site was observed for signs of swelling, discharge or wound dehiscence, and/or abscess.

(d) Post-Operative Analgesia:

A dose of 12.5 ug of fentanyl per hour was achieved by applying a 25 ug/hour patch with half of its drug delivery surface covered. This patch was applied to the dorsal surface of each rabbit prior to surgery to provide post-operative analgesia until Day 3. Tegaderm™ was placed over the patch to ensure adherence. Following surgery, an Elizabethan collar was placed on the animal to protect the patch. The patch and collar were removed 3 days following surgical procedures.

(e) Post-Operative Antibiotic:

Prophylactic post-operative antibiotics were not provided. However, Cavilon spray was applied topically to the incision site once post-operatively. If signs of infection were observed in any animal that animal was treated per veterinary instruction.

(f) Suture Removal:

Skin sutures were removed 12 days post-surgery.

Results:

(a) Clinical Animal Observations:

Following completion of the 10-day post-operative daily observation period, cageside observations were conducted every day before noon, including weekends and holidays. These observations confirmed the general health and viability of the animals, documented the availability of food and water, and included a qualitative assessment of food water input and urine/feces output.

All rabbits survived to scheduled termination.

No adverse findings or observations were noted during daily cageside observations. Overall, animals gained weight or showed normal minor weight fluctuations throughout the course of the study.

Pre-operative examinations including color of mucous membranes, heart rate, respiratory rate, and body temperature for all animals were within acceptable ranges (see Table below) with the exception that two (2) animals had a respiratory rate greater than the maximum target value and three (3) animals had body temperatures that were slightly lower than the target minimum.

TABLE 10
Target Values/Ranges for vital signs:

| Vital Signs | Target Values |
|---|---|
| Color of Mucosal membranes | Pink |
| Capillary refill time | 2 seconds |
| Heart rate | 130-325 beats per minute |
| Respiratory rate | 4-10 respirations per 10 (10) second count |
| Blood oxygen saturation | 90% |
| Body temperature | 38-40 C. |

In both instances in which respiratory rate was greater than the maximum target value these measurements were within range when data was recorded throughout surgical procedures. In instances in which body temperatures were low, the values were similar to many of the other animals in this study as well as greater when measurements were recorded at the first interval during surgical procedures suggesting that the pre-surgical body temperatures may have been spurious as body temperature typically decreases with anesthesia administration. Body temperatures for all animals decreased as surgical procedures progressed and were typically out of range upon arrival in post-operative recovery although heart rate, respiratory rate, blood oxygen saturation, and color of mucous membranes remained within acceptable ranges.

(b) Necropsy

All animals were euthanized and had necropsy performed at 39 days post-surgery. Rabbits were anesthetized with ketamine/xylazine and euthanized with an intracardiac dose of at least 150 mg/kg of sodium barbital.

Necropsy observations for each animal were limited to each administration site (femur) and the surrounding structures and left and right papliteal lymph nodes. The defect sites and the surrounding structures were grossly evaluated for healing and signs of inflammation or infection. Local tissue structures, including the adjacent synovial lining and joint surfaces were examined for inflammation or the presence of particulate debris. No signs of inflammation/infection were noted. No particulate debris was observed. No gross lesions were noted in tissues/organs not specified in the protocol for collection.

The administration sites (femora) and the left and right popliteal lymph nodes were collected, maintained separately, and stored in 10% neutral buffered formalin at ambient temperature. Femora were trimmed at the end proximal to the surgical site.

(c) Histology:

For all implants sites: foreign implanted material was visible with fibrosis and/or giant cells seen and containing and rimmed by bony trabeculae

Hematopoiesis was increased especially in the marrow spaces of the implants with HA and erythropoiesis was slightly emphasized although as many myeloid elements also seen. The exception was EOPO=HA 13-0082-06L. Hematopoiesis seemed one severity grade greater in the EOPO vs the PUTTY.

The only focus of inflammation not seen within the implant site is in PUTTY and the inflammation surrounding the foreign material was consistent with implant material and interpreted as a much earlier reaction to the implant lacking the fibrosis and bony trabeculae seen in the more mature implants.

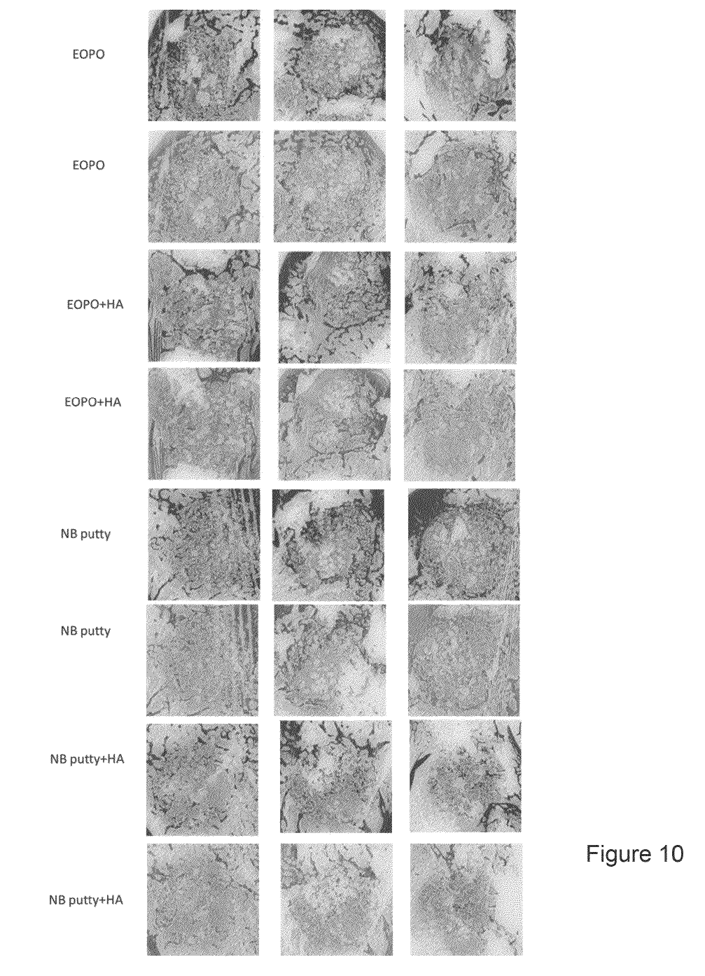

Detail histology results are described in the Table 11 below and shown in FIG. 10.

| | | | Question near filled | |
|---|---|---|---|---|
| | | | defect region and sur- | |
| ID | LEG | IMPLANT | rounding space | Results |
| 01 | R | EOPO | normal marrow? | Focal pocket of hem- |
| | | | | atopoiesis adjacent to |
| | | | | implant similar to |
| | | | | hematopoiesis densi- |
| | | | | ty in diaphysis (mar- |
| | | | | row normal) |
| 04 | L | EOPO | normal marrow? | Fibrosis, peri-implant- |
| | | | | 1; Roughly similar |
| | | | | amounts of hemato- |
| | | | | poiesis peri-implant |
| | | | | as in diaphysis (mar- |
| | | | | row normal) |
| 05 | L | EOPO | normal marrow? | Roughly similar |
| | | | | amounts of hemato- |
| | | | | poiesis peri-implant |
| | | | | as in diaphysis (mar- |
| | | | | row normal) |
| 02 | R | EOPO + HA | hypercellular marrow? If | Increased hemato- |
| | | | yes, why? Hematopoie- | poiesis in implant-4 |
| | | | sis or inflammation? | |
| 04 | R | EOPO + HA | hypercellular marrow? If | Increased hemato- |
| | | | yes, why? Hematopoie- | poiesis in implant-4; |
| | | | sis or inflammation? | focal pocket of hema- |
| | | | | topoiesis adjacent to |
| | | | | implant-3 |
| 06 | L | EOPO + HA | hypercellular marrow? If | Fibrosis, peri-implant- |
| | | | yes, why? Hematopoie- | 3; Roughly similar |
| | | | sis or inflammation? | amounts of hemato- |
| | | | | poiesis peri-implant |
| | | | | as in diaphysis and |
| | | | | no significant hema- |
| | | | | topoiesis in the im- |
| | | | | plant (marrow normal) |
| 01 | L | PUTTY | normal marrow? | Focal pocket of hem- |
| | | | | atopoiesis adjacent to |
| | | | | implant is roughly |
| | | | | similar to density of |
| | | | | that in diaphysis |
| | | | | (marrow normal) |
| 02 | L | PUTTY | normal marrow? | Fibrosis, peri-implant- |
| | | | | 2; Focal inflammation |
| | | | | with foreign material |
| | | | | close to implant-2; |
| | | | | Marrow normal |
| 03 | L | PUTTY | normal marrow? | Fibrosis, peri-implant- |
| | | | | 1; Marrow normal |
| 03 | R | PUTTY + HA | hypercellular marrow? If | Roughly similar |
| | | | yes, why? Hematopoie- | amounts of hemato- |
| | | | sis or inflammation? | poiesis peri-implant |
| | | | | as in diaphysis; In- |
| | | | | creased hematopoie- |
| | | | | sis in implant-1 |
| 05 | R | PUTTY + HA | hypercellular marrow? If | Increased hemato- |
| | | | yes, why? Hematopoie- | poiesis in implant-3 |
| | | | sis or inflammation? | |
| 06 | R | PUTTY + HA | hypercellular marrow? If | Roughly similar |
| | | | yes, why? Hematopoie- | amounts of hemato- |
| | | | sis or inflammation? | poiesis peri-implant |
| | | | | as in diaphysis; In- |
| | | | | creased hematopoie- |
| | | | | sis in implant-2 |

Summary:

Overall, animals gained weight or showed normal minor weight fluctuations throughout the course of the study.

No adverse findings or observations were noted during daily cageside observations performed post-surgery. Animals were sacrificed and necropsy was performed at 39 days post-surgery. All animals survived to scheduled termination. At sacrifice all grafted sites were grossly examined for defect filling. Remnant bone graft material and the regional lymph nodes were evaluated for any system toxicity via standard histologic methods. No gross observations were noted at necropsy.

### Example 3

An irrigation resistant matrix (IRM) consists of different amounts of variable diameter bioglass, poloxamer 124, poloxamer 407, and sodium hyaluronate. This study examines the effects of changing the ratios of the variable diameter bioglasses and poloxamers. After mixing 19 samples, compression and sustainability testing was performed.

The purpose of this study was to examine the effect of altering the ratios of different diameter bioglasses and poloxamers in IRM and to see if the resulting samples perform well under the compression and sustainability tests.

The samples varied in the amount of different diameter bioglass and poloxamers added. *HA was not added to any sample.

Table 12 provides the tested compositions.

TABLE 12
Glass and poloxamer amounts of IRM samples:

| | Sample | 1-2 | 90-710 | 32-125 | <90 | Poloxamer | Poloxamer | HA |
|---|---|---|---|---|---|---|---|---|
| Sample ID | Number | mm (g) | μm (g) | μm (g) | μm (g) | 124 (g) | 407 (g) | (g) |
| B | 1 | 13 | 13 | 19.5 | 19.5 | 17.5 | 17.5 | 0 |
| C | 2 | 13.34 | 13.34 | 20.01 | 20.01 | 19.98 | 13.32 | 0 |
| D | 3 | 14.2 | 14.2 | 21.3 | 21.3 | 17.4 | 11.6 | 0 |
| E | 4 | 14.8 | 14.8 | 22.2 | 22.2 | 13 | 13 | 0 |
| F | 5 | 13 | 13 | 19.5 | 19.5 | 14 | 21 | 0 |
| G | 6 | 23.03 | 23.03 | 19.74 | 0 | 13.68 | 20.52 | 0 |
| H | 7 | 24.85 | 24.85 | 21.3 | 0 | 14.5 | 14.5 | 0 |
| I | 8 | 24.85 | 24.85 | 21.3 | 0 | 17.4 | 11.6 | 0 |
| J | 9 | 32.5 | 16.25 | 16.25 | 0 | 14 | 21 | 0 |
| K | 10 | 35.2 | 17.6 | 17.6 | 0 | 14.8 | 14.8 | 0 |
| L | 11 | 31.75 | 25.41 | 9.54 | 0 | 13.32 | 19.98 | 0 |
| M | 12 | 33.8 | 27.05 | 10.15 | 0 | 14.5 | 14.5 | 0 |
| N | 13 | 37.4 | 20.4 | 10.2 | 0 | 12.8 | 19.2 | 0 |
| P | 14 | 25.34 | 25.34 | 21.72 | 0 | 11.04 | 16.56 | 0 |
| S | 15 | 0 | 56 | 13 | 0 | 12.4 | 18.6 | 0 |
| T-73 | 16 | 32.85 | 29.2 | 10.95 | 0 | 10.8 | 16.2 | 0 |
| T-70 | 17 | 31.5 | 28 | 10.5 | 0 | 12 | 18 | 0 |
| T-67 | 18 | 30.15 | 26.8 | 10.05 | 0 | 13.2 | 19.8 | 0 |
| 60-HA | 19 | 27 | 24 | 9 | 0 | 16 | 24 | 0.5* |

Description of Testing Equipment

i. IRM compression testing

ii. Force per displacement was measured from the Shimadzu Mechanical Strength Tester.

IRM Sustainability Testing

The samples were immersed PBS in small beakers and placed in the incubator shaker. Tests were conducted in the R&D analytical lab under ambient conditions between 25 and 28 degrees Celsius.

Compression testing: the force per displacement value was measured from displacement values of 0-4.0 mm starting after the force reached 1 Newton.

Sustainability testing: a scale was created to evaluate the IRM after sustainability testing.

Test Procedures:

Compression Testing:

a) Weigh out 7.5 g of the IRM sample.

b) Place sample into 10 cc syringe. Mold the IRM into a cylinder and expel from syringe using the compressed gas aerosol can.

c) Place the IRM cylinder into the Shimadzu Mechanical Strength Tester.

d) Record the force per displacement (i.e., slope of the compression graph from 0-4 mm).

Sustainability

a) The sample was immersed in PBS in a 250 cc beaker and left undisturbed for 5 minutes.

b) The beakers were placed into the incubator shaker for 3 minutes at 300 rpm and room temperature.

c) Once the cycle was finished the samples were removed from the shaker. A rating was assigned to each sample.

Samples 6-10 were prepared by mixing all bioglass for these samples together before adding to the poloxamers.

Evaluation

Compression testing was competed when total displacement was equal to 10 mm. There was no acceptance criterion for the IRM samples in the context of compression testing. Compression testing was conducted for investigative purposes.

Sustainability testing was completed after oscillation for three minutes was finished. Photographs of each sample were taken immediately following the oscillation cycle. Samples were accepted if the IRM remained a ball or scored at least a four on the rating scale.

Results

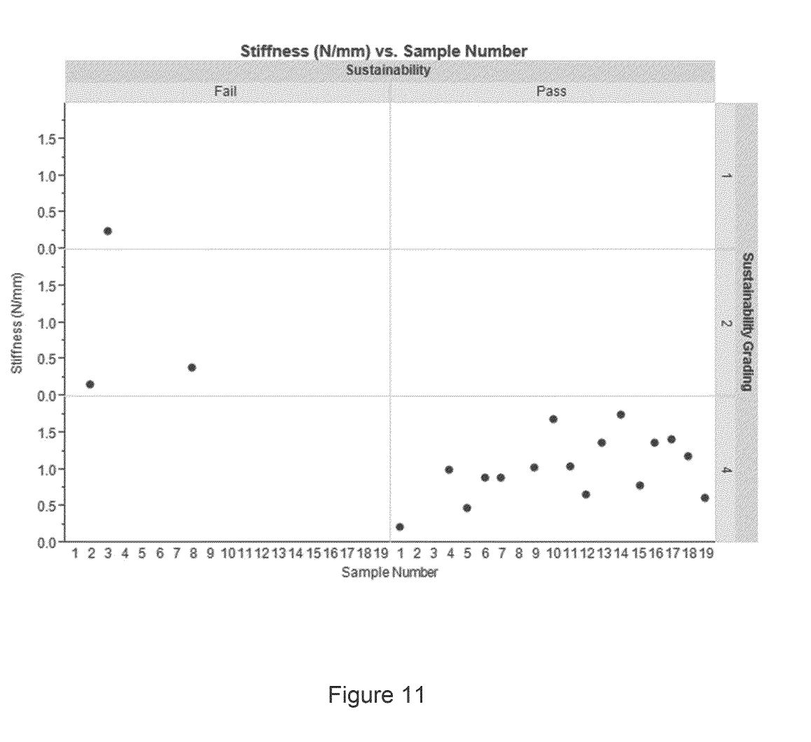

FIG. 11 shows compression and sustainability results for samples 1-19. There was a wide variety of stiffness values for the IRM samples. The control samples (samples 15-19) did not have a higher or lower value than the experimental groups.

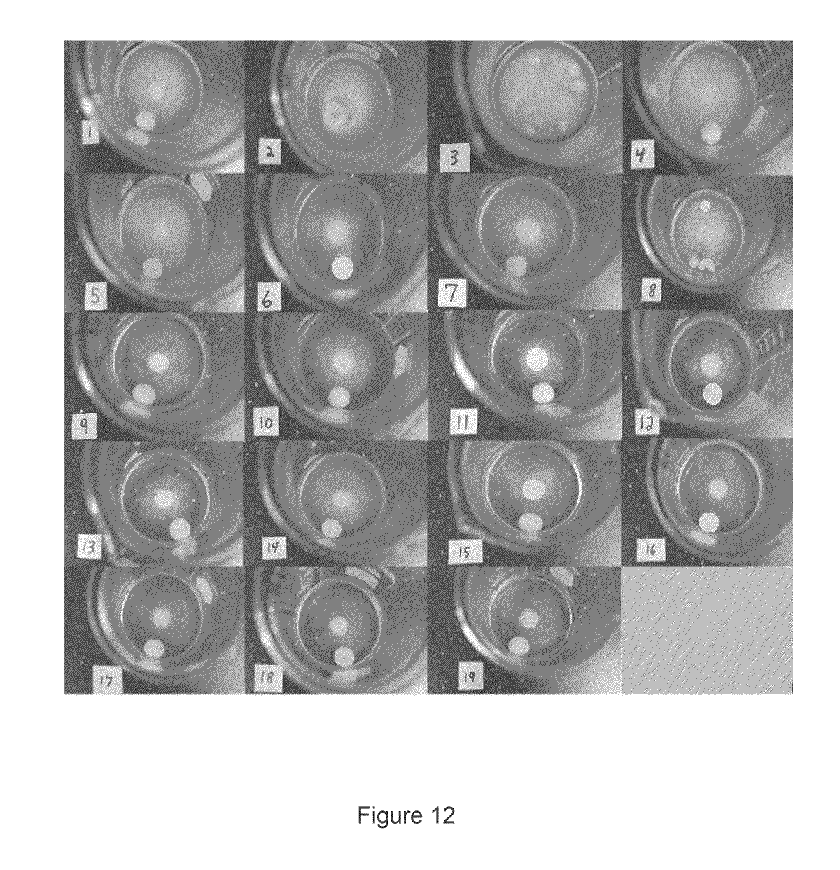

FIG. 12 shows sustainability testing of samples 1-19 (samples correspond with Table 9). Samples 2, 3, and 8 were the only samples that failed the sustainability test. The test was repeated and samples 2 and 4 were assigned a 2 and sample 3 was assigned a 1 under the rating scale as follows:

Immersion Sustainability Rating Scale

1: The ball of IRM has completely disintegrated. 0-33% of the original
sphere remains intact.
2: The ball of IRM has mostly disintegrated. 33-66% or less of the original
sphere is intact.
3: The ball of IRM has slightly disintegrated. Greater than 66% of the e
sphere is intact.
4: The ball of IRM has not disintegrated and has retained its shape. There
may be smaller granules of glass the escape the ball during stirring,
but the larger 1-2 mm diameter glass does not dissolve into the solution.

Discussion

The variations in bioglass percentages and poloxamer percentages caused a great deal of variation in the physical properties of IRM. There may be a correlation between a higher Force per Displacement value, and better performance in sustainability testing. This theory was suggested by the fact that the samples that failed the sustainability test tended to have lower Force per Displacement values. The samples that failed the sustainability test had a range of 0.1475-0.3871 N/mm Force per Displacement Value while all the samples had a range from 0.1475-1.6854 N/mm.

### CONCLUSIONS

Certain formulations may be preferable over others depending on the application at hand. If the IRM is, for example, is used in a syringe, a more moldable sample with lower Force per Displacement value could be used. For applications in where IRM may be needed to be quickly reabsorbed into the body, a formulation in where smaller diameter bioglass could be used.

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, are intended to define the spirit and scope of this invention.

1. An irrigation resistant bone repair composition comprising:
a biocompatible or bioactive bone repair material, and a mixture of at least one non-random poly(oxyalkylene) block copolymer and at least one non-ionic surfactant other than a non-random poly(oxyalkylene) block copolymer.

2. The bone repair composition of claim 1, wherein the poly(oxyalkylene) block copolymer is a poloxamer polymer.

3. The bone repair composition of claim 1, wherein the poly(oxyalkylene) block copolymer is selected from the group consisting of poloxamer 407, poloxamer 124, poloxamers 188, poloxamer 237, and poloxamer 338.

4. The bone repair composition of claim 1, wherein the weight ratio of the mixture is 1%-99% relative to the weight of the bone repair composition.

5. The bone repair composition of claim 1, wherein the weight ratio of the mixture is 1%-20% relative to the weight of the bone repair composition.

6. The bone repair composition of claim 1, wherein the weight ratio of the mixture is 20%-30% relative to the weight of the bone repair composition.

7. The bone repair composition of claim 1, wherein the weight ratio of the mixture is 30%-40% relative to the weight of the bone repair composition.

8. The bone repair composition of claim 1, wherein the weight ratio of the mixture is 40%-50% relative to the weight of the bone repair composition.

9. The bone repair composition of claim 1, wherein the weight ratio of the mixture is 50%-60% relative to the weight of the bone repair composition.

10. The bone repair composition of claim 1, wherein the weight ratio of the mixture is 60%-70% relative to the weight of the bone repair composition.

11. The bone repair composition of claim 1, wherein the weight ratio of the mixture is 70%-80% relative to the weight of the bone repair composition.

12. The bone repair composition of claim 1, wherein the weight ratio of the mixture is 80%-99% relative to the weight of the bone repair composition.

13. The bone repair composition of claim 1, wherein the weight ratio of the poly(oxyalkylene) block copolymer to the weight ratio of the at least one non-ionic surfactant is from about 1% to 99%.

14. The bone repair composition of claim 1, wherein the weight ratio of the poly(oxyalkylene) block copolymer to the weight ratio of the at least one surfactant is about 50% to 50%.

15. The bone repair composition of claim 1, wherein the weight ratio of the poly(oxyalkylene) block copolymer to the weight ratio of the at least one surfactant is about 99% to 1%.

16. The bone repair composition of claim 1, wherein the composition is osteoconductive.

17. The bone repair composition of claim 1, wherein the composition is osteostimulative.

18. The bone repair composition of claim 1, wherein the bone repair material is a bioactive glass or ceramic.

19. The bone repair composition of claim 18, wherein the bioactive glass is melt-derived bioactive glass or sol-gel derived bioactive glass.

20. The bone repair composition of claim 19, wherein the bioactive glass is in the form of a particle.

21. The bone repair composition of claim 20, wherein the bioactive glass particle comprises about 15-45% CaO, about 30-70% SiO₂, about 0-25% Na₂O, about 0-17% P₂O₅, about 0-10% MgO and about 0-5% CaF₂.

22. The bone repair composition of claim 20, wherein the bioactive glass particle comprises about 45% SiO₂, about 24.5% CaO, about 6% P₂O₅, and about 2.5% Na₂O.

23. The bone repair composition of claim 20, wherein the size of the bioactive glass particle is in a range from about 0.01 um to about 5 mm.

24. The bone repair composition of claim 20, wherein the bioactive glass comprises 0-80% 1000-2000 um bioactive glass, 0-90% 90-710 um bioactive glass, and 0-90% 32-125 um bioactive glass.

25. The bone repair composition of claim 20, wherein the bone repair material is one or more particles of bioactive glass coated with a glycosaminoglycan, wherein the glycosaminoglycan is bound to the bioactive glass.

26. The bone repair composition of claim 25, wherein the glycosaminoglycan is selected from the group consisting of heparin, heparan sulfate, chondroitin sulfate, dermatan sulfate, keratan sulfate, and hyaluronic acid.

27. The bone repair composition of claim 1, further comprising at least one element selected from the group consisting of Li, K, Mg, Sr, Ti, Zr, Fe, Co, Cu, Zn, Al, Ag, Ga, P, N, S, F, Cl, and I.

28. The bone repair composition of claim 20, wherein the bioactive glass particle is pretreated in a solution comprising one or more of blood, bone marrow, bone marrow concentrate, bone-morphogenetic proteins, platelet-rich plasma, and osteogenic proteins.

29. The bone repair composition of claim 28, wherein the proteins are selected from the group consisting of WP9QY(W9), OP3-4, RANKL, B2A, P1, P2, P3, P4, P24, P15, TP508, OGP, PTH, NBD, CCGRP, W9, (Asp)₆, (Asp)₈, and (Asp, Ser, Ser)₆, and mixtures thereof.

30. The bone repair composition of claim 1, wherein the composition is in a form of a putty, paste, gel, or waxy solid.

31. The bone repair composition of claim 1, wherein the composition, when implanted into a surgical site, maintains position and does not displace upon irrigation of the surgical site.

32. The bone repair composition of claim 1, wherein the non-ionic surfactants is selected from the group consisting of

33. The bone repair composition of claim 1, wherein the non-ionic surfactant other than the non-random poly(oxyalkylene) block copolymer is selected from the group consisting of fatty alcohols, alkoxylated alcohols, alkoxylated alkylphenols, alkoxylated fatty amides, alkoxylated fatty esters, alkoxylated fatty ethers, alkoxylated sorbitan esters, alkoxylated sorbitan esters, fatty acids, fatty acid esters, polyol esters, and polyalkylene glycols.

34. An irrigation resistant putty or paste including the composition of claim 1 mixed with water, saline, blood, or BMA.

35. The bone repair composition of claim 1, wherein the composition is for treating a bone defect or a bone gap.

36. The bone repair composition claim 1, wherein the composition is for regeneration of hard tissues.

37. A method for treating a bone having a bone gap or a bone defect comprising contacting the bone at or near the site of the bone defect with the bone repair composition of claim 1.

38. A kit comprising:
at least one tube comprising the bone repair composition of claim 1,
a dispensing gun,
an adapter, and
optionally, at least one dispensing tip.

39. The kit of claim 38, wherein the tube comprising the bone repair composition is capped.

40. The kit of claim 38, further comprising a syringe.

41. The kit of any of claim 38, further comprising at least one of “Y” connector, tube connector, and an aspiration needle.
